(19) United States
(12) Patent Application Publication
(10) Pub. No.: US 2023/0049115 A1
(43) Pub. Date: Feb. 16, 2023

(54) SYSTEM AND METHOD FOR TARGET THERMAL ANALYSIS IN COMPLEX FLUIDS

(71) Applicant: Portland State University, Portland, OR (US)

(72) Inventors: Albert S. Benight, Milwaukie, OR (US); Matthew W. Eskew, Portland, OR (US)

(73) Assignee: Portland State University, Portland, OR (US)

(21) Appl. No.: 17/815,154

(22) Filed: Jul. 26, 2022

Related U.S. Application Data
(60) Provisional application No. 63/203,660, filed on Jul. 27, 2021.

(51) Int. Cl. G01N 33/557; G01N 33/569; G01N 33/53; G01N 33/68

(52) U.S. Cl. CPC G01N 33/557; G01N 33/569; G01N 33/5308; G01N 33/6845

(57) ABSTRACT

Methods for detecting, identifying, and/or quantifying a target molecule in a complex fluid using thermal analysis are disclosed. Exemplary complex fluids include biofluids and environmental fluids. Exemplary target molecules include proteins, peptides, nucleic acids, lipids, carbohydrates, viruses, and combinations thereof. A method for using thermal analysis to determine whether purification affects one or more characteristics, such as binding characteristics, of a target molecule is also disclosed.

### CROSS REFERENCE TO RELATED APPLICATION

This application claims the benefit of the earlier filing date of U.S. Provisional Application No. 63/203,660, filed Jul. 27, 2021, which is incorporated by reference in its entirety herein.

### FIELD

This disclosure concerns a system and method for target molecule thermal analysis in complex fluids.

### SEQUENCE LISTING

The nucleic and amino acid sequences are shown using standard letter abbreviations for nucleotide bases, and three letter code for amino acids, as defined in 37 C.F.R. § 1.822. Only one strand of each nucleic acid sequence is shown, but the complementary strand is understood as included by any reference to the displayed strand.

The Sequence Listing is submitted as an XML file, “Sequence.xml,” created on Jul. 26, 2022, 8192 bytes, which is incorporated by reference herein.

### SUMMARY

This disclosure concerns embodiments of a system and method for target thermal analysis in complex fluids. In some embodiments, an analysis method includes (a) obtaining an analysis sample comprising a quantity of a complex fluid, the complex fluid comprising one or more of proteins, peptides, lipids, and carbohydrates, the complex fluid further comprising or suspected of comprising a target molecule; (b) obtaining an analysis sample thermogram by differential scanning calorimetry; (c) inputting the analysis sample thermogram into a computer system; (d) comparing, using the computer system, the analysis sample thermogram to a (i) control sample thermogram, the control sample comprising the complex fluid, the control sample being devoid of the target molecule, (ii) one or more reference library thermograms of samples comprising known target molecules in the complex fluid, or both (i) and (ii) to provide a comparison; e) determining, using the computer system and based at least in part on the comparison, whether the analysis sample thermogram exhibits a perturbation; and (f) if a perturbation is present, identifying the target molecule as present in the complex fluid.

In any of the foregoing or following embodiments, the target molecule may comprise a protein, peptide, nucleic acid, lipid, carbohydrate, virus, or any combination thereof. In any of the foregoing or following embodiments, the perturbation may comprise (i) a change in height and/or width of a peak on the analysis sample thermogram relative to a corresponding peak on the control sample thermogram or reference library thermogram; or (ii) a shift in position of a peak on the analysis sample thermogram relative to a corresponding peak on the control sample thermogram or reference library thermogram; or (iii) presence of a peak on the analysis sample thermogram that is not present on the control sample thermogram or reference library thermogram; or (iv) absence of a peak on the analysis sample thermogram compared to a peak that is present on the control sample thermogram or reference library thermogram; or (v) any combination of (i), (ii), (iii), and (iv).

In one implementation, the method further includes determining a mass of the target molecule in the complex fluid. In another implementation, the method further includes adding a quantity of a ligand to the analysis sample and determining whether the ligand binds to the target molecule.

In yet another implementation, the method further includes combining a quantity of a ligand with the analysis sample prior to obtaining the analysis sample thermogram, the ligand capable of binding to the target molecule; and comparing, using the computer system, the analysis sample thermogram to (i) a control sample thermogram, the control sample comprising the complex fluid and the ligand, the control sample being devoid of the target molecule, (ii) a reference library of thermograms of samples comprising the ligand, or both (i) and (ii) to provide the comparison.

In still another implementation, the method further comprises combining an additive with the analysis sample to provide a modified analysis sample, the additive comprising an inorganic salt, a protein, a carbohydrate, an amino acid, a vitamin, a peptide, a fatty acid, a lipid, a therapeutic agent, a solvent, or any combination thereof; obtaining a modified analysis sample thermogram; inputting the modified analysis sample thermogram into the computer system; comparing, using the computer system, the modified analysis sample thermogram to the analysis sample thermogram; and determining, using the computer system and based at least in part on the comparison, whether the modified analysis sample thermogram exhibits a perturbation relative to the analysis sample thermogram, wherein a perturbation indicates that the additive (i) altered a structure of the target molecule, (ii) altered an interaction of the ligand, if present, with the target molecule, or (iii) both (i) and (ii).

In any of the foregoing or following embodiments, the analysis sample may be obtained by combining, in a vessel, a capture moiety and a complex fluid comprising a target molecule or suspected of comprising a target molecule, the capture moiety comprising biotin covalently attached to a ligand capable of binding to the target molecule; incubating the complex fluid and capture moiety whereby the target molecule, if present, binds to the capture moiety to form a conjugate; removing the conjugate, if present, from the complex fluid with a device comprising (i) a body comprising a substrate material, a poly(methyl methacrylate) (PMMA) coating on at least a portion of a surface of the body, and a plurality of retrieval moiety molecules covalently bound to the PMMA coating, the retrieval moiety molecules comprising streptavidin; removing the target molecule from the device; and combining the removed target molecule with a quantity of a control sample to provide the analysis sample, wherein the control sample comprises the complex fluid, the control sample being devoid of the target molecule.

In some embodiments, the complex fluid comprises the target molecule in an unpurified state, the control sample comprises the complex fluid and the target molecule in a purified state, and a perturbation indicates that the target molecule in the purified state has an altered characteristic compared to the target molecule in the unpurified state. In certain implementations, the method further includes combining a quantity of a ligand with the analysis sample prior to obtaining the analysis sample thermogram, the ligand capable of binding to the target molecule in the unpurified state; comparing, using the computer system to a control sample thermogram, the control sample comprising the complex fluid, the target molecule added in the purified state, and the ligand to provide a subsequent comparison; and determining, using the computer system and based at least in part on the subsequent comparison, whether the analysis sample exhibits a perturbation, wherein a perturbation indicates that the target molecule in the unpurified state exhibits different binding characteristics to the ligand compared to the target molecule in the purified state.

In certain implementations, the analysis sample is obtained from a subject; the analysis sample comprises a quantity of a complex fluid comprising one or more of proteins, peptides, lipids, and carbohydrates, the complex fluid further comprising or suspected of comprising a virus; the analysis sample thermogram is compared to a (i) control sample thermogram, the control sample comprising the complex fluid, the control sample being devoid of the virus, (ii) one or more reference library thermograms of samples comprising known viruses in the complex fluid, or both (i) and (ii) to provide a comparison; and a perturbation indicates the virus is present in the complex fluid. The method may further include identifying the virus based at least in part on the perturbation, the subsequent perturbation, or the one or more genomic or structural analyses.

### BRIEF DESCRIPTION OF THE DRAWINGS

FIGS. 1A and 1B are a front perspective view and a side view, respectively, of an exemplary capture device.

FIG. 2 shows exemplary DNA probe sequences useful as standard molecules—SEQ ID NOS: 1-5.

FIG. 3 illustrates a generalized example of a suitable computing environment in which described embodiments, techniques, and technologies pertaining to target thermal analysis can be implemented.

FIGS. 4A-4D show results of streptavidin-biotin binding in reconstituted E. coli lysate; FIG. 4A is a thermogram of 2 mg/mL purified E. coli lysate standard; FIG. 4B shows thermograms of 2 mg/mL E. coli lysate spiked with streptavidin to mimic 33% protein expression—2 mg/mL E. coli lysate standard (▪), 2 mg/mL E. coli lysate with 1 mg/mL streptavidin (•), 2 mg/mL E. coli lysate and 1 mg/mL streptavidin with 2 molar equivalents of biotin (▴); FIG. 4C shows thermograms of 1 mg/mL E. coli lysate spiked with streptavidin to mimic 33% protein expression—2 mg/mL E. coli lysate standard (▪), 1 mg/mL E. coli lysate with 0.5 mg/mL streptavidin (•), 1 mg/mL E. coli lysate and 0.5 mg/mL streptavidin with 2 molar equivalents of biotin (▴); FIG. 4D shows thermograms of 2 mg/mL E. coli lysate spiked with streptavidin to mimic ˜11% protein expression—2 mg/mL E. coli lysate standard (▪), 2 mg/mL E. coli lysate with 0.25 mg/mL streptavidin (•), 2 mg/mL E. coli lysate and 0.25 mg/mL streptavidin with 2 molar equivalents of biotin.

FIGS. 5A-5D are difference thermograms for streptavidin and streptavidin-biotin mixtures— FIG. 5A shows the residual curves obtained when the thermogram for E. coli lysate (2 mg/mL) was subtracted from the thermogram of E. coli lysate (2 mg/mL)+streptavidin (1 mg/mL) (1), and E. coli lysate (2 mg/mL)+streptavidin (1 mg/mL)+2 molar equivalents of biotin (2); FIG. 5B shows the residual curves when the thermogram for E. coli lysate (1 mg/ml) alone was subtracted from the thermograms of the E. coli lysate (1 mg/mL)+streptavidin (0.5 mg/mL) (1); and the thermograms of E. coli lysate (1 mg/mL)+streptavidin (0.5 mg/mL)+2 molar equivalents biotin (2); FIG. 5C shows the residual curves when the thermogram for E. coli lysate (2 mg/ml) alone was subtracted from the thermograms of the E. coli lysate (2 mg/mL)+streptavidin (0.25 mg/mL) (1); and the thermograms of E. coli lysate (2 mg/mL)+streptavidin (0.25 mg/mL)+2 molar equivalents biotin (2); FIG. 5D shows the thermograms for streptavidin (1 mg/mL) (1) and streptavidin (1 mg/mL)+2 molar equivalents biotin (2) in buffer (shift of streptavidin thermogram (1) with biotin binding (2) similarly displayed a ˜20% decrease in peak height as also seen in FIGS. 5A-5C).

FIGS. 6A-6C are thermograms for angiotensin converting enzyme (ACE2)—FIG. 6A shows thermograms for the HEK293 cell supernatant background (▪) and a solution of the supernatant containing an unknown amount of expressed ACE2 (•); FIG. 6B is the difference thermogram corresponding to ACE2 protein alone obtained by subtracting the curves in FIG. 6A; FIG. 6C is the measured thermogram for purified ACE2 in standard PBS buffer.

FIGS. 7A-7C are thermograms of ACE2 with ligands lisinopril and captopril—FIG. 7A shows thermograms of the cell culture supernatant background (▪); expressed ACE2 in the supernatant (•); expressed ACE2 in the supernatant plus captopril (▴) or lisinopril (▾); FIG. 7B shows differences of the thermograms in FIG. 7A after subtraction of the thermogram of the supernatant background. ACE2 (▪); ACE2+captropril (•); ACE2+lisinopril (▴); FIG. 7C shows differences of the difference curves in FIG. 7B obtained after subtracting the curve for ACE2 alone from those for captopril (▪) and lisinopril (•). These curves indicate changes to the ACE2 thermogram strictly due to effects of the ligand (captopril or lisinopril) interactions with ACE2.

FIGS. 8A-8B are thermograms of the receptor-binding domain (RBD) of COVID-19—FIG. 8A shows thermograms of purified RBD alone in buffer (•), unpurified ACE2 in the supernatant background (▪), and RBD added to added unpurified ACE2 in the supernatant background (▴); FIG. 8B shows difference thermograms obtained by subtracting the thermograms of unpurified ACE2 in the supernatant and RBD alone in buffer from the composite thermogram measured for the mixtures of unpurified ACE2 plus RBD (▴); ACE2 plus captopril (▪), ACE2+lisinopril (•). Note: the difference curves for captopril and lisinopril were reproduced from FIG. 7C.

FIGS. 9A and 9B are transition heat capacity curves for human serum albumin (HSA) and lysozyme, respectively, at the Tₘ.

FIG. 10 is a DNA hairpin transition heat capacity curve at the Tₘ for a 20-base single strand oligomer (SEQ ID NO: 6).

FIGS. 11A-11C are thermograms for angiotensin converting enzyme (ACE2)—FIG. 11A shows thermograms for an HEK293 cell supernatant background (▪) and a solution of the supernatant containing an unknown amount of expressed ACE2 (•); FIG. 11B shows the difference thermogram corresponding to ACE2 protein alone obtained by subtracting the curves in FIG. 11A (▪) and the thermogram for purified ACE2 (•); FIG. 11C is the baseline fitted curve of the difference thermogram from FIG. 11B; the inset in FIG. 11C is a published reference thermogram for bovine somatic ACE2 (Voronov et al., FEBS Letters 2002, 522:77-82).

FIG. 12 is a flowchart of an exemplary general process for building a database.

FIG. 13 is a flowchart of an exemplary machine learning model.

FIG. 14 is a flowchart of an exemplary process for scoring complex fluid samples.

FIG. 15 is a flowchart of an exemplary process for solubilizing drug samples.

FIG. 16 is a flowchart of an exemplary process for screening unpurified proteins.

FIG. 17 is a flowchart of an exemplary process for infectious disease analysis.

### DETAILED DESCRIPTION

Embodiments of a method for detecting, identifying, and/or quantifying target molecules in a complex fluid are disclosed. In some embodiments, the method further includes capturing the target molecule. Target molecules may be detected, identified, quantified, captured, purified, analyzed, or any combination thereof by embodiments of the disclosed method. Advantageously, target molecule purification is not required prior to performing embodiments of the disclosed method. The disclosure also encompasses kits comprising standard molecules for use with embodiments of the disclosed methods.

Production of biopharmaceuticals (biologics—e.g., proteins, peptides, antibodies, antibody fragments, and other small molecules) and low abundance proteins is accomplished using prokaryotic or eukaryotic expression systems. This allows for manufacturing a large array of small quantity proteins for screening applications and large scale applications, on the metric-ton scale, for specific therapeutic applications, such as vaccine production. An attractive feature of using protein expression systems is that it is possible to produce this wide variety of biological compounds (proteins, peptides, antibodies) on various scales, in a cheap and rapid manner. A classic example of a biologic produced at large scale is insulin. However, a major drawback is that while expression of the targeted biological compounds is cheap and fast, isolation and purification of expressed molecules can be expensive and time consuming. For instance, conventional purification strategies for a moderately abundant and soluble protein can require 27 individual steps and four days to complete (Wingfield, Current Protocols in Protein Science 2014, 78:6.2.1-6.2.22). Candidate biologics also must be screened and tested for efficacy, bioavailability, and/or ADMET (absorption, distribution, metabolism, excretion, and toxicity) properties. However, unlike small molecule drug discovery, biologics must first be expressed and purified before being tested. The added costs for preparation, testing, and screening of a therapeutic biologic can greatly increase research and development expenditures compared to the standard small molecule drug discovery pipeline. Biologics also present unique challenges in the preclinical drug discovery pipeline. The uncertain and potentially unique nature of how biologics function in the body present additional challenges limiting their development. Standard testing of compounds in animal models is at best marginally effective. Complications can arise because biologic compounds tend to target specific receptors or epitopes; and toxicological information does not necessarily translate between species. As a result, only a few primate species have proven to be viable testing models (Baumann, Current Drug Metabolism 2006, 7:15-21). Therefore, early pharmacokinetic/pharmacodistribution (PK/PD) information is incredibly important for guiding toxicological studies and human risk assessment. However, this becomes an issue when each lead compound must be expressed and purified before determining binding affinity to a targeted receptor.

Targeted biologic products of an expression system are expressed in culture media. In initial stages of the preparation process, cells harboring the over-expressed target are lysed releasing the target into a crude cell lysate. Unpurified crude lysates containing over-expressed proteins are produced at the beginning of the preparation process of a biologic. In the generic process, an expression system contains a constructed coding element (plasmid or other suitable vector) that codes for the desired target biologic. Cells containing the vector that produce the biologic target are grown in culture until cells can no longer grow or reproduce and expire. Harvested cells are centrifuged, resuspended in buffer, and lysed. The genetic material (DNA/RNA) is then removed through extraction and centrifugation. At this stage, depending on the biologic, the desired expressed unpurified protein can reside in either the lysed cellular media or in the culture supernatant. Conventionally, it is at this stage that the laborious and multi-step purification process begins; usually a process that must be completed before meaningful biochemical and biological testing of overexpressed compounds can be performed.

Some embodiments of the disclosed method may be used to screen and detect expressed proteins, such as biologic products and low abundance proteins, in unpurified cell lysates from both prokaryotic and eukaryotic cell systems. Advantageously, the method is applicable at early stages of biologic production and circumvents the necessity for isolation and purification of the expressed protein target. Additionally, in some implementations, the method is useful for investigating ligand binding to, and interactions with, unpurified proteins in crude cell lysates. Advantageously, embodiments of the disclosed process utilizes the signal produced by differential scanning calorimetry (DSC) as a tool for detection of ligand binding to expressed proteins in the complex fluids, such as prokaryotic and eukaryotic liquid culture expression systems. The consequences of this advancement are huge. Biologics target specific receptors based on structure. If purification alters this structure, then the biologic will show a lack of efficacy. Likewise, if designing a target molecule that binds to a ligand, evaluation with a purified ligand may provide a false negative and invalidate an otherwise suitable drug compound. Embodiments of the disclosed method also are useful for measuring protein-protein interactions in unpurified systems.

Some implementations of the disclosed method are useful for detecting and analyzing viral outbreaks. In some examples, the method may be used to capture and/or analyze viruses from complex fluids. Viruses display distinctly characteristic thermograms. Unique thermograms for viruses are thought to arise because of the unique array of proteins that typically decorate viral surfaces (Orlov et al., FEBS Letters 1998, 433:307-311). Thus, the presence of viral infections may be detected from abnormal plasma thermograms or thermograms of individual viral coat proteins.

Currently, other than culture and amplification (both costly and time intensive) there are no methods available for accurate and rapid screening of biofluids for the presence of active virus. However, with the presence of virus particles in diseased biofluids or convalescent plasma comes the expectation that thermograms of infected samples will be significantly different compared to the normal thermogram. Thus, embodiments of the disclosed method may be used as potentially very sensitive indicators of viral infection. In some instances, the presence of virus particles in plasma can be unequivocally confirmed using a capture moiety as disclosed herein. The actual physical isolation of virus particles directly from plasma is a far more accurate indicator of active infection than detection of remnants of viral RNA using PCR.

Embodiments of the disclosed method are useful for diagnostics relating to virtually any disease, viral or otherwise. Advantageously, there is complete non-reliance on viral genome sequences, or antibodies against viral or other infectious agents, for accurate diagnoses of infected samples.

### I. General Considerations, Definitions and Abbreviations

The following explanations of terms and abbreviations are provided to better describe the present disclosure and to guide those of ordinary skill in the art in the practice of the present disclosure. As used herein, “comprising” means “including” and the singular forms “a” or “an” or “the” include plural references unless the context clearly dictates otherwise. Further, the term “coupled” encompasses mechanical, electrical, magnetic, optical, as well as other practical ways of coupling or linking items together, and does not exclude the presence of intermediate elements between the coupled items. The term “or” refers to a single element of stated alternative elements or a combination of two or more elements, unless the context clearly indicates otherwise. Furthermore, as used herein, the term “and/or” means any one item or combination of items in the phrase.

Unless explained otherwise, all technical and scientific terms used herein have the same meaning as commonly understood to one of ordinary skill in the art to which this disclosure belongs. Although methods and materials similar or equivalent to those described herein can be used in the practice or testing of the present disclosure, suitable methods and materials are described below. The materials, methods, and examples are illustrative only and not intended to be limiting. Other features of the disclosure are apparent from the following detailed description and the claims.

The disclosure of numerical ranges should be understood as referring to each discrete point within the range, inclusive of endpoints, unless otherwise noted. Unless otherwise indicated, all numbers expressing quantities of components, molecular weights, percentages, temperatures, times, and so forth, as used in the specification or claims are to be understood as being modified by the term “about.” Accordingly, unless otherwise implicitly or explicitly indicated, or unless the context is properly understood by a person of ordinary skill in the art to have a more definitive construction, the numerical parameters set forth are approximations that may depend on the desired properties sought and/or limits of detection under standard test conditions/methods as known to those of ordinary skill in the art. When directly and explicitly distinguishing embodiments from discussed prior art, the embodiment numbers are not approximates unless the word “about” is recited.

Although there are alternatives for various components, parameters, operating conditions, etc. set forth herein, that does not mean that those alternatives are necessarily equivalent and/or perform equally well. Nor does it mean that the alternatives are listed in a preferred order unless stated otherwise.

The systems, methods, and apparatus described herein should not be construed as being limiting in any way. Instead, this disclosure is directed toward all novel and non-obvious features and aspects of the various disclosed embodiments, alone and in various combinations and subcombinations with one another. The disclosed systems, methods, and apparatus are not limited to any specific aspect or feature or combinations thereof, nor do the disclosed things and methods require that any one or more specific advantages be present or problems be solved. Furthermore, any features or aspects of the disclosed embodiments can be used in various combinations and subcombinations with one another.

Although the operations of some of the disclosed methods are described in a particular, sequential order for convenient presentation, it should be understood that this manner of description encompasses rearrangement, unless a particular ordering is required by specific language set forth below. For example, operations described sequentially can in some cases be rearranged or performed concurrently. Moreover, for the sake of simplicity, the attached figures may not show the various ways in which the disclosed things and methods can be used in conjunction with other things and methods. Additionally, the description sometimes uses terms like “analyze,” “apply,” “build,” “determine” “display,” “estimate,” “generate,” “identify,” “instruct,” “obtain”, “produce,” “receive”, “train” to describe the disclosed computer-implemented methods. Such terms are high-level abstractions of the actual operations that are performed. The actual operations that correspond to these terms can vary depending on the particular implementation and can be readily discerned by one of ordinary skill in the art.

Theories of operation, scientific principles, or other theoretical descriptions presented herein in reference to the apparatus or methods of this disclosure have been provided for the purposes of better understanding and are not intended to be limiting in scope. The apparatus and methods in the appended claims are not limited to those apparatus and methods that function in the manner described by such theories of operation.

Definitions of common terms in chemistry may be found in Richard J. Lewis, Sr. (ed.), Hawley's Condensed Chemical Dictionary, published by John Wiley & Sons, Inc., 2016 (ISBN 978-1-118-13515-0). Definitions of common terms in molecular biology may be found in Benjamin Lewin, Genes VII, published by Oxford University Press, 2000 (ISBN 019879276X); Kendrew et al. (eds.), The Encyclopedia of Molecular Biology, published by Blackwell Publishers, 1994 (ISBN 0632021829); and Robert A. Meyers (ed.), Molecular Biology and Biotechnology: a Comprehensive Desk Reference, published by Wiley, John & Sons, Inc., 1995 (ISBN 0471186341); and other similar references.

In order to facilitate review of the various embodiments of the disclosure, the following explanations of specific terms are provided:

Additive: As used herein, the term “additive” refers to a component added to a complex fluid. Additives include, but are not limited to, proteins, inorganic salts, buffering systems, carbohydrates, amino acids, vitamins, peptides, fatty acids, lipids, trace elements, heavy metals, antibiotics, and combinations thereof.

Biofluid: A fluid obtained from a biological organism. Exemplary biofluids include, but are not limited to blood, blood components (e.g., plasma, serum) mucus, cerebrospinal fluid, sputum, urine, tears, and sweat. The term biofluid also includes extracted and modified biofluids, e.g., CAR (chimeric antigen receptor) T cells, platelet-rich plasma, and the like.

Compartment: Body fluids (biofluids) typically found in the human or other animal bodies are arranged into five major body compartments including, blood plasma, interstitial fluid, fat tissues, lymph, intracellular fluids, and transcellular fluids. As defined by Rang and Dales Pharmacology, 8^{th} edition, Churchill Livingstone, 2016.

Complex fluid: As used herein, the term complex fluid refers to a fluid including one or more of proteins, peptides, lipids, carbohydrates, or any combination thereof. The term complex fluid encompasses biofluids, environmental fluids, cell culture supernatants, cell lysates, and in vitro (prokaryotic and eukaryotic) expression systems, regardless of origin, strain, or species.

Conjugate: Two or more moieties directly or indirectly coupled together. For example, a first moiety may be covalently or noncovalently (e.g., electrostatically) coupled to a second moiety. Indirect attachment is possible, such as by using a “linker” (a molecule or group of atoms positioned between two moieties).

Differential scanning calorimetry (DSC): DSC measures the difference in the amount of heat required to raise the temperature of a sample and a reference as a function of temperature. During a phase transition, such as when a protein “melts” or unfolds, the amount of heat required changes, thereby providing a characteristic melting curve (ΔCₚ (kcal/mol⋅° C.) vs temperature (° C.)) of the protein as temperature is increased, where Cₚ is specific heat for a constant pressure process. The temperature at which the phase transition occurs varies from protein to protein. When a ligand binds to the protein, the melting temperature (mid-point of the melting curve) and/or maximum ΔCₚ may be affected.

Dumbbell-shaped double hairpin loop: A nucleic acid structure with two spaced-apart hairpin loops separated by a region of base pairs:

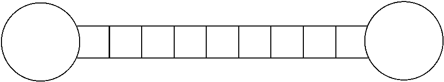

Environmental fluid: Water or an environmentally derived solvent, with or without contaminants (cellular, chemical, and/or physical).

Expression system: A prokaryotic or eukaryotic expression system may include a cell culture comprising prokaryotic or eukaryotic cells to which a vector (plasmid, transposon, etc.) that is coded for the biological compound(s) of interest is added. Targeted compounds are produced until cells are unable to survive or reproduce anymore. The cells are then lysed and genetic material (DNA/RNA) is removed through centrifugation. At this stage, depending on the properties of the targeted compound, the unpurified protein can be found in either the lysed cellular media or in the culture supernatant.

Hairpin loop: An unpaired loop in DNA or RNA (e.g., mRNA) that forms when a nucleic acid strand folds and forms base pairs with another section of the same strand, resulting in a loop of unpaired bases, e.g.:

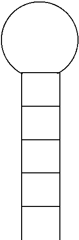

Ligand: As used herein, the term “ligand” refers to a molecule that binds to a target molecule. The ligand may be an exogenous or endogenous molecule that binds to a target molecule, such as a plasma protein.

Moiety: A moiety is a fragment of a molecule, or a portion of a conjugate.

Perturb/perturbation: As used herein, the terms perturb, perturbed, and perturbation refer to differences (e.g., peak shifts, peak height/area variations) between a sample thermogram and a control thermogram.

Polymer: A molecule of repeating structural units (e.g., monomers) formed via a chemical reaction, i.e., polymerization.

Probe: A substance used to detect or identify another substance in a sample.

Small molecule: As used herein, the term “small molecule” refers to an active agent (a drug medicament, pharmaceutical, therapeutic agent, nutraceutical, or other compound) having a molecular weight of less than 1,000 daltons.

Soluble: Capable of becoming molecularly or ionically dispersed in a solvent to form a homogeneous solution. U.S. Pharmacopeia definitions: very soluble: more than 1000 mg/ml, freely soluble: 100-1000 mg/ml, soluble: 30-100 mg/ml, sparingly soluble: 10-30 mg/ml, slightly soluble: 1-10 mg/ml, very slightly soluble: 0.1-1 mg/ml, practically insoluble or insoluble: <0.1 mg/ml.

Subject: An animal (human or non-human) subjected to a treatment, observation or experiment. Includes both human and veterinary subjects, including human and non-human mammals, such as rats, mice, cats, dogs, pigs, horses, cows, and non-human primates.

Target molecule: A molecule of interest generally a macromolecule or macromolecular assembly. Exemplary target molecules include, but are not limited to, naturally occurring and/or non-naturally occurring proteins, peptides, nucleic acids, lipids, phospholipids, carbohydrates, and viruses.

Therapeutic agent: A compound that may be administered to a subject to effect a change, such as treatment, amelioration, or prevention of a disease or disorder or at least one symptom associated therewith.

Thermogram: As used herein, the term “thermogram” refers to a melting curve of a complex fluid sample, the thermogram produced by differential scanning calorimetry

### II. Target Thermal Analysis in Complex Fluids

This disclosure concerns embodiments of methods for detecting, identifying, and/or quantifying a target molecule in a complex fluid using thermal analysis. Complex fluids may include biofluids and environmental fluids. Exemplary target molecules include, but are not limited to, proteins, peptides, nucleic acids, lipids, carbohydrates, viruses, and combinations thereof Also disclosed is a method for using thermal analysis to determine whether purification affects one or more characteristics, such as binding characteristics, of a target molecule.

A method for target thermal analysis may include (a) obtaining an analysis sample comprising a quantity of a complex fluid, the complex fluid comprising one or more of proteins, peptides, lipids, and carbohydrates, the complex fluid further comprising or suspected of comprising a target molecule; (b) obtaining an analysis sample thermogram by differential scanning calorimetry; (c) inputting the analysis sample thermogram into a computer system; (d) comparing, using the computer system, the analysis sample thermogram to a (i) control sample thermogram, the control sample comprising the complex fluid, the control sample being devoid of the target molecule, (ii) one or more reference library thermograms of samples comprising known target molecules in the complex fluid, or both (i) and (ii) to provide a comparison; I determining, using the computer system and based at least in part on the comparison, whether the analysis sample thermogram exhibits a perturbation; and (f) if a perturbation is present, identifying the target molecule as present in the complex fluid. In some embodiments, the reference library thermogram is of a sample comprising a known quantity of the known target molecule in the complex fluid. In some embodiments, the comparison is a “difference thermogram” obtained by subtracting the control sample or reference library thermogram from the analysis sample thermogram. The computer system may be used to produce the difference thermogram.

In any of the foregoing or following embodiments, the target molecule may comprise a protein, peptide, nucleic acid, lipid, carbohydrate, virus, or any combination thereof. In one implementation, the target is naturally occurring. In another implementation, the target is non-naturally occurring. In some examples, the target molecule comprises a virus or a viral protein. Exemplary viruses include, but are not limited to, the SARS-CoV-2 virus (COVID 19). In some implementations, the target molecule is a biological compound produced by a prokaryotic or eukaryotic expression system. When encountered insoluble targets (proteins, peptides, etc.) can be analyzed in aqueous or complex environments.

In any of the foregoing or following embodiments, the comparing may comprise subtracting the control sample thermogram or the one or more reference library thermograms from the analysis sample thermogram. In any of the foregoing or following embodiments, the perturbation may comprise (i) a change in height and/or width of a peak on the analysis sample thermogram relative to a corresponding peak on the control sample thermogram or reference library thermogram; or (ii) a shift in position of a peak on the analysis sample thermogram relative to a corresponding peak on the control sample thermogram or reference library thermogram; or (iii) presence of a peak on the analysis sample thermogram that is not present on the control sample thermogram or reference library thermogram; or (iv) absence of a peak on the analysis sample thermogram compared to a peak that is present on the control sample thermogram or reference library thermogram; or (v) any combination of (i), (ii), (iii), and (iv).

When a perturbation is present, the method may further include determining a mass of the target molecule in the complex fluid by (i) adding a known amount of the target molecule or a standard molecule to the control sample to provide a known sample; obtaining a known sample thermogram; inputting the known sample thermogram into the computer system; comparing, using the computer system, the known sample thermogram to the control sample thermogram; determining, based at least in part on the comparison, a known perturbation measurement corresponding to the known amount of the target molecule or the standard molecule; comparing the known perturbation measurement to a perturbation measurement corresponding to the target molecule in the analysis sample to provide a measurement comparison; and determining, based at least in part on the measurement comparison, an amount of the target molecule in the analysis sample.

In another implementation when a perturbation is present, the method further comprises determining an amount of the target molecule in the complex fluid by: determining, using the computer system and based at least in part on the perturbation, a perturbation measurement of the target molecule; comparing, using the computer system, the thermodynamic melting parameter of the target molecule to a calibration curve comprising perturbation measurements of calibration solutions comprising varying amounts of a standard molecule, to provide a measurement comparison; and determining, using the computer system and based at least in part on the measurement comparison, an amount of the target molecule in the analysis sample. In some implementations, the perturbation may correspond to a thermodynamic melting parameter. The calibration curve may be prepared by preparing a plurality of calibration solutions, each solution comprising an amount of a standard molecule; obtaining a calibration thermogram of each calibration solution; inputting the calibration thermogram of each calibration solution into the computer system; determining, using the computer system, a perturbation measurement of each calibration solution; and constructing, using the computer system, a calibration curve of perturbation measurements versus standard molecule amounts.

In some embodiments, the method further includes adding a quantity of a ligand to the analysis sample; obtaining a subsequent analysis sample thermogram by differential scanning calorimetry; inputting the subsequent analysis sample thermogram into the computer system; comparing, using the computer system, the subsequent analysis sample thermogram to the analysis sample thermogram to provide a subsequent comparison; and determining, using the computer system and based at least in part on the subsequent comparison, whether the subsequent analysis sample thermogram exhibits a subsequent perturbation, wherein a subsequent perturbation indicates binding of the ligand to the target molecule. In some implementations, the method further comprises determining, based at least in part on the perturbation, a characteristic of an interaction of the ligand with the target molecule, wherein the characteristic is a binding constant, reaction enthalpy, binding stoichiometry, binding free energy, binding entropy, or any combination thereof.

In some implementations, a quantity of ligand capable of binding to the target molecule is combined with the analysis sample prior to obtaining the analysis sample thermogram, and the method further includes comparing, using the computer system, the analysis sample thermogram to (i) a control sample thermogram, the control sample comprising the complex fluid and the ligand, the control sample being devoid of the target molecule, (ii) a reference library of thermograms of samples comprising the ligand, or both (i) and (ii) to provide the comparison. If a perturbation is present, the method may further comprise determining an amount of the target molecule in the complex fluid by: adding a known amount of a standard molecule to the control sample to provide a known sample; obtaining a known sample thermogram; inputting the known sample thermogram into the computer system; comparing, using the computer system, the known sample thermogram to the control sample thermogram; determining, based at least in part on the comparison, a known perturbation measurement corresponding to the known amount of the target molecule or the standard molecule; comparing the known perturbation measurement to a perturbation measurement corresponding to the target molecule in the analysis sample to provide a measurement comparison; and determining, based at least in part on the measurement comparison, an amount of the target molecule in the analysis sample.

In some embodiments, the complex fluid comprises the target molecule in an unpurified state and the control sample comprises the fluid complex and the target molecule in a purified state. In such implementations, a perturbation indicates that the target molecule in the purified state has an altered characteristic compared to the target molecule in the unpurified state. In certain implementations, when the target molecule is in an unpurified state, the method further comprises combining a quantity of a ligand with the analysis sample prior to obtaining the analysis sample thermogram, the ligand capable of binding to the target molecule in the unpurified state; comparing, using the computer system to a control sample thermogram, the control sample comprising the complex fluid, the target molecule added in the purified state, and the ligand to provide a subsequent comparison; and determining, using the computer system and based at least in part on the subsequent comparison, whether the analysis sample exhibits a perturbation, wherein a perturbation indicates that the target molecule in the unpurified state exhibits different binding characteristics to the ligand compared to the target molecule in the purified state.

In any of the foregoing or following embodiments, when a ligand is present and a perturbation is found, the method may further include determining, based at least in part on the perturbation, a characteristic of an interaction of the ligand with the target molecule, wherein the characteristic is a binding constant, reaction enthalpy, binding stoichiometry, binding free energy, binding entropy, or any combination thereof.

In some implementations, an additive is combined with the analysis sample prior to obtaining the analysis sample thermogram, and the method further includes obtaining a modified analysis sample thermogram; inputting the modified analysis sample thermogram into the computer system; comparing, using the computer system, the modified analysis sample thermogram to the analysis sample thermogram; and determining, using the computer system and based at least in part on the comparison, whether the modified analysis sample thermogram exhibits a perturbation relative to the analysis sample thermogram, wherein a perturbation indicates that the additive (i) altered a structure of the target molecule, (ii) altered an interaction of the ligand, if present, with the target molecule, or (iii) both (i) and (ii). Exemplary additives include, but are not limited to, an inorganic salt, a protein, a carbohydrate, an amino acid, a vitamin, a peptide, a fatty acid, a lipid, a therapeutic agent, a solvent, or any combination thereof.

In any of the foregoing or following embodiments, obtaining the analysis sample may further comprise: combining, in a vessel, a capture moiety and a complex fluid comprising a target molecule or suspected of comprising a target molecule, the capture moiety comprising biotin covalently attached to a ligand capable of binding to the target molecule; incubating the complex fluid and capture moiety whereby the target molecule, if present, binds to the capture moiety to form a conjugate; removing the conjugate, if present, from the complex fluid with a device comprising (i) a body comprising a substrate material, a poly(methyl methacrylate) (PMMA) coating on at least a portion of a surface of the body, and a plurality of retrieval moiety molecules covalently bound to the PMMA coating, the retrieval moiety molecules comprising streptavidin; removing the target molecule from the device; and combining the removed target molecule with a quantity of a control sample to provide the analysis sample, wherein the control sample comprises the complex fluid, the control sample being devoid of the target molecule.

In an independent embodiment, a method comprises combining, in a vessel, (i) a complex fluid, comprising one or more of proteins, peptides, lipids, and carbohydrates, the complex fluid further comprising or suspected of comprising a target molecule, and (ii) a capture moiety comprising biotin covalently attached to a ligand capable of binding to the target molecule; incubating the complex fluid and capture moiety whereby the target molecule, if present, binds to the capture moiety to form a conjugate; removing the conjugate, if present, from the complex fluid with a device comprising (i) a body comprising a substrate material, a poly(methyl methacrylate) (PMMA) coating on at least a portion of a surface of the body, and a plurality of retrieval moiety molecules covalently bound to the PMMA coating, the retrieval moiety molecules comprising streptavidin; removing the target molecule from the device; combining the removed target molecule with a quantity of a control sample to provide an analysis sample, wherein the control sample comprises the complex fluid, the control sample being devoid of the target molecule; obtaining an analysis sample thermogram by differential scanning calorimetry; inputting the analysis sample thermogram into a computer system; comparing, using the computer system, the analysis sample thermogram to (i) a control sample thermogram, the control sample comprising the complex fluid and the ligand, the control sample being devoid of the target molecule, (ii) a reference library of thermograms of samples comprising the ligand, or both (i) and (ii) to provide a comparison; determining, using the computer system and based at least in part on the comparison, whether the analysis sample thermogram exhibits a perturbation; and if a perturbation is present, identifying the target molecule as present in the complex fluid.

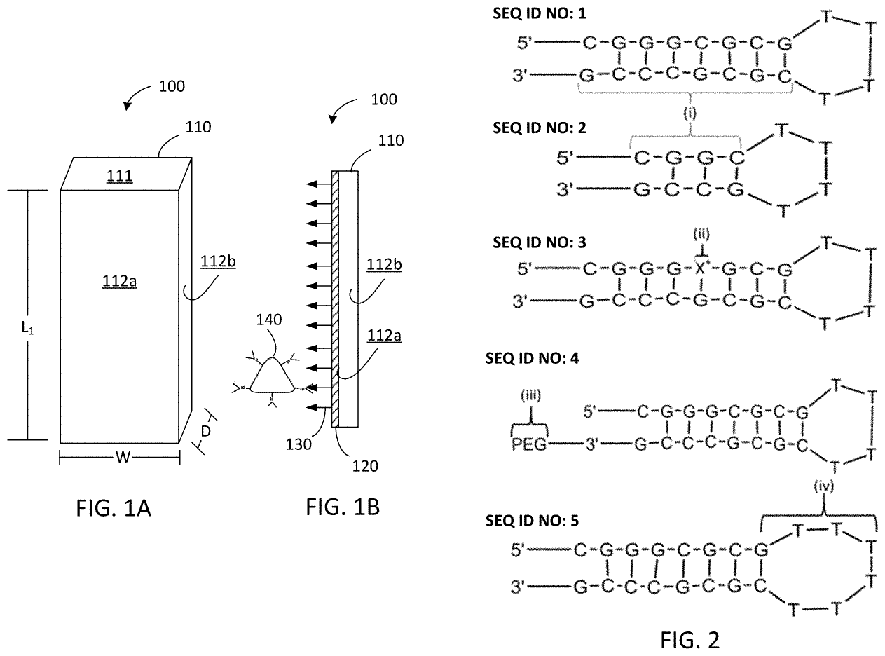

In some embodiments, as shown in FIG. 1A, a capture device 100 comprises an elongated body 110 having a length L1 and a polygonal cross-section orthogonal to the length L. The body 100 has an upper surface 111, a lower surface (not visible in FIG. 1A), and a plurality of side surfaces 112a, 112b, etc. Although the exemplary body 110 of FIG. 1A has a rectangular cross-section (see, e.g., upper surface 111), it is understood that the cross-section may be any polygon including three or more sides, e.g., a triangle, square, rectangle, parallelogram, trapezoid, pentagon, hexagon, octagon, or the like. Alternatively, the cross-section may be cylindrical or elliptical. As shown in FIG. 1B, the capture device 100 further includes a poly(methyl methacrylate) (PMMA) coating 120 on at least a portion of a surface (e.g., surface 112a) of the body 110, and a plurality of retrieval moiety molecules 130 bound to at least a portion of the PMMA coating 120. In some embodiments, the retrieval moiety comprises streptavidin molecules. The PMMA may be functionalized, e.g., by exposure to O2 plasma, to create carboxylic groups to which streptavidin is subsequently attached. In any of the foregoing embodiments, a plurality of capture moieties 140 may be bound to at least some of the retrieval moieties 130. In certain embodiments, the capture moieties comprise a biotinylated protein, such as biotinylated human serum albumin (HSA). Additional disclosure regarding suitable capture devices may be found in US 2022/0091128 A1, which is incorporated by reference herein in its entirety.

In some examples, the target molecule comprises a virus or a viral protein. When the complex fluid or analysis sample is obtained from a subject, identifying the virus or viral protein as present in the complex fluid indicates the subject has a viral infection. In such embodiments, the analysis sample is obtained from the subject and comprises a quantity of a complex fluid comprising one or more of proteins, peptides, lipids, and carbohydrates, the complex fluid further comprising or suspected of comprising a virus. The analysis sample thermogram is compared to a (i) control sample thermogram, the control sample comprising the complex fluid, the control sample being devoid of the virus, (ii) one or more reference library thermograms of samples comprising known viruses in the complex fluid, or both (i) and (ii) to provide a comparison. A perturbation indicates the virus is present in the complex fluid.

In one implementation where a perturbation is found, the method further includes: adding a quantity of a ligand to the analysis sample to provide a subsequent analysis sample; obtaining a subsequent analysis sample thermogram by differential scanning calorimetry; inputting the subsequent analysis sample thermogram into the computer system; comparing, using the computer system, the subsequent analysis sample thermogram to the analysis sample thermogram; and determining, using the computer system and based at least in part on the comparison, whether the subsequent analysis sample thermogram exhibits a subsequent perturbation, wherein a subsequent perturbation indicates binding of the ligand to the virus.

In another implementation where a perturbation is found, the method further includes: combining a capture moiety with the analysis sample, the capture moiety comprising biotin covalently attached to a ligand capable of binding to the virus; incubating the complex fluid and capture moiety whereby the virus binds to the capture moiety to form a conjugate; removing the conjugate, if present, from the complex fluid with a device comprising (i) a body comprising a substrate material, a poly(methyl methacrylate) (PMMA) coating on at least a portion of a surface of the body, and a plurality of retrieval moiety molecules covalently bound to the PMMA coating, the retrieval moiety molecules comprising streptavidin; and removing the virus from the device.

After removing the virus from the device, the method may further comprise performing one or more genomic of structural analyses of the virus. In another embodiment, after removing the virus from the device, the method further comprises screening a ligand or a capture moiety to determine whether the ligand or capture moiety is capable of binding to the virus. Screening may comprise: combining the virus with a complex fluid devoid of the virus to provide a subsequent analysis sample; adding a quantity of the ligand or capture moiety to the subsequent analysis sample; obtaining a subsequent analysis sample thermogram by differential scanning calorimetry; inputting the subsequent analysis sample thermogram into the computer system; comparing, using the computer system, the subsequent analysis sample thermogram to a control sample thermogram, the control sample comprising the complex fluid and the virus, the control sample being devoid of the ligand or capture moiety; and determining, using the computer system and based at least in part on the comparison, whether the subsequent analysis sample thermogram exhibits a subsequent perturbation, wherein a subsequent perturbation indicates binding of the ligand or capture moiety to the virus. In some embodiments, the ligand or capture moiety comprises a therapeutic agent or therapeutic agent candidate.

In any of the foregoing embodiments where the target molecule is a virus or viral protein, the method may further comprise identifying the virus based at least in part on the perturbation, the subsequent perturbation, or the one or more genomic or structural analyses.

In any of the foregoing or following embodiments, the complex fluid may be a biofluid, an environmental fluid, or any combination thereof. In some embodiments, the biofluid comprises plasma, serum, mucus, cerebrospinal fluid, sputum, urine, cell culture fluid, a cell lysate, a cell culture supernatant, growth media, or any combination thereof. In certain implementations, the complex fluid is a biofluid comprising plasma, serum, mucus, cerebrospinal fluid, sputum, urine, or any combination thereof. Growth media may contain any number or combination of proteins, inorganic salts, buffering systems, carbohydrates, amino acids, vitamins, peptides, fatty acids, lipids, trace elements, antibiotics, etc. necessary for the growth of an expression system. Exemplary environmental fluids include, but are not limited to, water (e.g., wastewater, river/stream water, lake/pond water, seawater, and the like) and environmentally-derived non-aqueous solvents.

In any of the foregoing or following embodiments, the standard molecule may be an external standard or an internal standard, and may have a different composition than the target molecule. In some implementations, the standard molecule comprises a nucleic acid, a protein, a peptide, or a modified molecule. Advantageously, the standard molecule has a thermogram with a peak outside of the complex media melting domain, thereby minimizing interference with the complex media. By knowing the mass of the standard molecule and the thermogram response it is possible to calculate the mass of a component from relative comparisons of thermogram characteristics (peak height, Tm (transition melting temperature), area under the curve, etc.,). For example, the standard molecule may be a nucleic acid probe comprising DNA or RNA, wherein the nucleic acid probe has a thermogram that is structurally distinct from a thermogram of the complex fluid. The thermogram response may be tuned by varying one or more combinations of primary and secondary structural features of the references probe, e.g., size, shape, and/or added base/backbone modifications. In some implementations, a nucleic acid probe comprises (i) two or more base pairs, or (ii) a modification that alters nucleic acid melting thermodynamics relative to an unmodified nucleic acid probe, or (iii) both (i) and (ii). In some examples, the nucleic acid probe comprises four or more base pairs. In certain implementations, the DNA or RNA may form a loop, such as a hairpin loop or a dumbbell-shaped double hairpin loop. For example, the DNA or RNA may include a self-complementary region, allowing it to form an intramolecular hairpin structure having a duplex region joined on one end (or both ends) by a single-strand loop (e.g., Vallone et al., Original Research on Biomolecules 1999, 50:425-442). Advantageously, DNA hairpins display well-defined two-state melting behavior, thus facilitating analysis. In contrast, protein probes may display peak broadening and/or non-two-state complications. Additionally, or alternatively, the DNA or RNA may be modified to tailor properties, e.g., by including stem and loop features and/or by PEGylation. In some embodiments the sequence is modified to provide a range of transition melting temperatures from less than 30° C. to greater than 90° C., such as from 25° C. to 95° C., 30° C. to 90° C., or 50° C. to 80° C. This range allows for the probes to be designed to minimize overlap with the DSC signal of the complex media background. Initial results have shown that there is no significant binding interaction between single or double stranded DNA and human serum albumin (HSA), a common ligand transport protein. However, the possibility exists that small DNA/RNA probes may interact with other target molecules. In this case, the probes may be modified with external modifications to alter the binding properties of the probe. External modifications include addition of polyethyleneglycol (PEG) or other generic modified structural components. These include backbone and base modifications. FIG. 2 shows several exemplary DNA probes with possible modifications including: (i) adjustable backbone length and/or sequence, and (ii) non-matching or non-Watson-Crick base pairs; additional backbone modifications can include: thymine dimer, covalent modifications, crosslinking, or bulged base-pairs represented by (X*); (iii) terminal modifications including PEGylation, secondary loop (dumbbell), phosphorylation, or any chemical modification may be introduced to tailor stability and binding properties of the DNA; (iv) loop size and/or base modifications. The size, sequence, number, and/or orientation are all tunable loop parameters that can potentially effect stability. In some embodiments, the functionally designed probes, or standard molecules, allow for a standard curve of thermogram response and probe mass to be constructed, enabling accurate determination of individual component masses in a complex fluid. Advantageously, transition heat capacities of proteins and DNA are essentially identical Thus, DNA standards are suitable for use with both nucleic acid and protein-based target molecules.

This disclosure also encompasses kits comprising a standard molecule as disclosed herein. In some embodiments, the standard molecule is a DNA or RNA probe. The probe may include a hairpin loop or a dumbbell-shaped double hairpin loop. In some embodiments, the probe is provided in a lyophilized state, providing shelf stability at room temperature. An end user can add a desired quantity of the lyophilized probe to a control or sample complex fluid for analysis. In some implementations, the kit further includes a quantity of a buffer to facilitate forming a solution having a known probe concentration. Aliquots of the probe solution can then be added to a control or sample complex fluid for analysis. The kit may further include instructions for using the DNA or RNA probe, optionally including instructions for preparing a probe solution.

In any of the foregoing or following embodiments, the ligand may comprise a protein, a peptide, a nucleic acid, a lipid, a carbohydrate, an organic small molecule having a molecular weight less than 1,000 daltons, a salt, an anion, a cation, a chelate, or any combination thereof. In one implementation, the ligand is naturally occurring. In another implementation, the ligand is non-naturally occurring. In some embodiments, the ligand comprises an organic small molecule therapeutic agent, an antibody, a CAR (chimeric antigen receptor) T cell, a nucleic acid probe, a CRISPR (clustered regularly interspaced short palindromic repeats) product, or any combination thereof. Insoluble ligands can be analyzed as long as the receptor compound is soluble in solution.

Embodiments of the disclosed methods and/or kits can be used for many purposes. Advantageously, the disclosed methods and/or kits can be used to detect, quantify, and/or identify target molecules in complex solutions without prior purification. Embodiments of the disclosed methods may be used with differential scanning calorimeter (DSC instrument). A standard molecule, e.g., a nucleic acid standard as disclosed herein may be used to calibrate the DSC instrument and/or determine the instrument resolution. Additionally, any data reduction method (e.g., pseudo-weighted end point methods, general sigmoidal fits, or fitting with multi-component models) to fit the thermogram baseline may be used with the disclosed processes and standard molecules, so long as the same measurement and analysis procedures are applied to both the standard and target molecules of interest.

In one embodiment, the method is used to detect presence of a target in a complex solution. In an independent embodiment, the method is used to detecting binding in complex solutions (e.g., binding of a ligand to a target molecule). In another independent embodiment, the method is used to determine chemical conditions for target molecule expression in a complex fluid. In other independent embodiments, the method is used to evaluate binding constants, thermodynamics, and/or relative populations of binding interactions in complex fluids. In still another independent embodiment, the method is used to determine structural and/or thermodynamic stabilities of proteins in complex fluids, e.g., by comparing a “difference thermogram” (obtained by subtracting a control sample thermogram of the complex fluid devoid of the target molecule from the sample analysis thermogram) with that of the purified protein alone. In yet another independent embodiment, the method is used to determine a mass of a target protein or proteins in a complex solution, e.g., by a relative intensity comparison to the added protein as a known concentration. In another independent embodiment, the method is used to determine a mass of a target protein or proteins in a complex fluid by comparison to a thermogram comprising a standard molecule (e.g., a DNA, RNA, protein, peptide, or other modified molecule). In still another embodiment, the method may be used, in combination with a capture device, to capture and retrieve analytes from complex fluids. In yet another independent embodiment, the method may be used to determine structural changes induced by purification of proteins expressed in complex fluids, thereby identifying changes that could yield inadvertent false-negative ligand binding results. In still another independent embodiment, the method may be used to determine a level of contamination in an environmental fluid and/or to identify contaminants in the environmental fluid. Advantageously, the disclosed methods are more precise than optical turbidity measurements and also can be used to measure physical stability of components in the environmental fluid compared to a background or standard sample. In another independent embodiment, the method may be used to determine expression and analysis of proteins generated from extremophile bacteria in extremophile environments. In still another independent embodiment, disclosed method may be used for rapid and accurate determination of drug-ligand binding constants and stoichiometry; additionally, the drug-ligand binding of aqueous insoluble drug compounds can be analyzed without the use of organic solvents and/or with milligram quantities of the drug compounds. Advantageously, drug-ligand binding can be assessed in complex fluids without isolation and purification of the target molecules, e.g., in expression systems; the binding can be evaluated in unpurified lysates in the complex fluid with detection of expressed proteins (e.g., antibodies, etc.) at concentrations in the μg/mL to mg/mL range.

Embodiments of the above-disclosed method are founded on the thermodynamic relationship that exists between mass, partial specific volume (PSV), and maximum calorimetric heat capacity (peak height on differential scanning calorimetry (DSC) thermograms) for target molecules, such as biomolecules. Additionally, each biomolecule has a unique DSC thermogram (peak height(s) and Tₘ(s)) that can be used to specifically identify a particular molecule in complex media. In some embodiments, the method includes using a standard molecule, e.g., a nucleic acid probe, such as a DNA probe. Advantageously, the transition heat capacities of DNA and proteins are essentially the same (differ by less than 1%). The maximum peak height on thermograms measured for specially designed and well-characterized DNA standard molecules, measured as a function of mass concentration, provides a calibration curve. Some DNA standard molecules are self-complementary oligomer sequences designed to form intramolecular hairpin structures that exhibit specific thermogram responses (i.e. Tₘ). Comparison of the thermogram peak height of a protein target of unknown mass with the standard curve for DNA enables determination of the unknown protein mass.

calorimetric responses for most proteins depend primarily on the mass of protein. Differences in transition heat capacity of individual proteins is essentially negligible (Johnson, Arch Biochem Biophys 2013; 531:100-109; Kholodenko et al., Anal Biochem 1999, 270:336-338; Gomez et al., Proteins: Structure, Function, and Bioinformatics 1995, 22:404-412). Experiments have shown that differences in observed calorimetric peak heights between different proteins and DNA oligonucleotides can be explained by differences in PSV (Eskew et al., Biochemical and Biophysical Research Communications 2022). In general, for proteins, it has been reported that a fundamental relationship exists between mass, transition heat capacity, PSV, and absolute heat capacity (peak height on DSC thermograms) (Kholodenko et al., Anal Biochem 1999, 270:336-338):

Cₚ^{max′}=(C_{p,p}−PSVₚ)·ρₚ (1)

cₚ^{max′}, is the maximum calorimetric peak height (measured absolute heat capacity at Tₘ) from the thermogram, C_{p,p}, transition heat capacity of the protein, PSVₚ the partial specific volume of the molecule, and ρₚ is the density of protein in solution. The protein in equation 1 can be substituted for any biomolecule (protein, peptide, antibody, oligonucleotide, etc.). Since the measured peak height on the DSC thermogram of proteins corresponds to the maximum heat capacity Cₚ^{max′}, it can be used to evaluate C_{p,p} for proteins (Id.; Gomez et al., Proteins: Structure, Function, and Bioinformatics 1995, 22:404-412). Likewise, C_{p,DNA} can also be found from Cₚ^{max′}. Further, there are negligible differences in the transition heat capacities C_{p,p} for different proteins (Id.) Values of C_{p,DNA} and C_{p,p} are also essentially equivalent (Eskew et al., bioRxiv 2021).

The same scheme can also be applied to DNA. Since thermograms are not normalized for mass or molecular weight, the same process is applied to determine the observed transition heat capacity for DNA with a slight modification of equation (1),

Cₘ^{max′}=(C_{p,DNA}−PSV_{DNA})·_{DNA} (2)

If the protein target and DNA molecule had the same transition heat capacity and PSV, then the measured calorimetric peak heights would be the same (Id.) While there is a relatively large difference in PSVₚ for DNA (0.54-0.60) and globular proteins (0.70-0.75), differences in transition heat capacities for DNA and proteins are negligible once normalized by mass.

Because transition heat capacities for proteins and DNA are essentially identical (per mass); rearranging the terms in equations 1 and 2 transition heat capacities for proteins and DNA are obtained:

$$for protein : {C}_{p , p} = \frac{{c}_{p , p}^{{max}^{′}}}{{ρ}_{p}} + {PSV}_{p} ; for DNA : {C}_{p , DNA} = \frac{{c}_{p , DNA}^{{max}^{′}}}{{ρ}_{DNA}} + {PSV}_{DNA} , \qquad ( 3 )$$

setting them equal to each other, since C_{p,p}=C_{p,DNA},

$$\frac{{c}_{p , p}^{{max}^{′}}}{{ρ}_{p}} + {PSV}_{p} = \frac{{c}_{p , DNA}^{{max}^{′}}}{{ρ}_{DNA}} + {PSV}_{DNA} \qquad ( 4 )$$

Equation 4 can then be rearranged to solve for the unknown mass concentration of the protein:

$${ρ}_{p} = \frac{{c}_{p , p}^{{max}^{′}}}{\frac{{c}_{p , DNA}^{{max}^{′}}}{{ρ}_{DNA}} + ( {PSV}_{DNA} - {PSV}_{p} )} \qquad ( 5 )$$

Equation 5 enables determination of the protein mass from the calibration curve, where C_{p,DNA}^{max′}/ρ_{DNA} is simply the slope of the best fit line. Since differences in transition heat capacity between proteins are small, observed differences in thermogram peak heights are predominantly due to differences in PSVₘₒₗ of individual molecules.

### III. Example Computing Environment

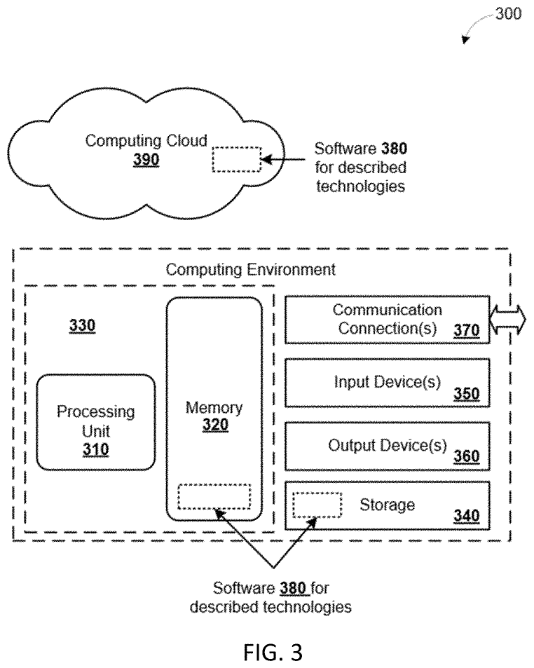

FIG. 3 illustrates a generalized example of a suitable computing environment 300 in which described examples, techniques, and technologies, including for determining identities or quantities of target molecules in a sample, can be implemented. For example, the computing environment 300 can implement all of the computer-implemented functions described herein.

The computing environment 300 is not intended to suggest any limitation as to scope of use or functionality of the technology, as the technology can be implemented in diverse general-purpose or special-purpose computing environments. For example, the disclosed technology can be implemented with other computer system configurations, including hand held devices, multiprocessor systems, microprocessor-based or programmable consumer electronics, network PCs, minicomputers, mainframe computers, and the like. The disclosed technology can also be practiced in distributed computing environments where tasks can be performed by remote processing devices that can be linked through a communications network. In a distributed computing environment, program modules can be located in both local and remote memory storage devices.

With reference to FIG. 3, the computing environment 300 includes at least one central processing unit 310 and memory 320. In FIG. 3, this most basic configuration 330 is included within a dashed line. The central processing unit 310 executes computer-executable instructions and can be a real or a virtual processor. In a multi-processing system, multiple processing units execute computer-executable instructions to increase processing power and, as such, multiple processors can be running simultaneously. The memory 320 can be volatile memory (e.g., registers, cache, RAM), non-volatile memory (e.g., ROM, EEPROM, flash memory, etc.), or some combination of the two. The memory 320 stores software 380, images, and video that can, for example, implement the technologies described herein. A computing environment can have additional features. For example, the computing environment 300 includes storage 340, one or more input devices 350, one or more output devices 360, and one or more communication connections 370. An interconnection mechanism (not shown) such as a bus, a controller, or a network, interconnects the components of the computing environment 300. Typically, operating system software (not shown) provides an operating environment for other software executing in the computing environment 300, and coordinates activities of the components of the computing environment 300. The terms computing environment, computing node, computing system, and computer are used interchangeably.

The storage 340 can be removable or non-removable, and includes magnetic disks, magnetic tapes or cassettes, CD-ROMs, CD-RWs, DVDs, or any other medium which can be used to store information and that can be accessed within the computing environment 300. The storage 340 stores instructions for the software 380 and measurement data, which can implement technologies described herein.

The input device(s) 350 can be a touch input device, such as a keyboard, keypad, mouse, touch screen display, pen, or trackball, a voice input device, a scanning device, or another device, which provides input to the computing environment 300. The input device(s) 350 can also include interface hardware for connecting the computing environment to control and receive data from host and client computers, storage systems, or administrative consoles.

For audio, the input device(s) 350 can be a sound card or similar device that accepts audio input in analog or digital form, or a CD-ROM reader that provides audio samples to the computing environment 300. The output device(s) 360 can be a display, printer, speaker, CD-writer, or another device that provides output from the computing environment 300.

The communication connection(s) 370 enable communication over a communication medium (e.g., a connecting network) to another computing entity. The communication medium conveys information such as computer-executable instructions, compressed graphics information, video, or other data in a modulated data signal.

Some examples of the disclosed methods can be performed using computer-executable instructions implementing all or a portion of the disclosed technology in a computing cloud 390. For example, a primary filesystem can be in the computing cloud 390, while a disclosed file index can be operated in the computing environment.

Computer-readable media are any available media that can be accessed within a computing environment 300. By way of example, and not limitation, with the computing environment 300, computer-readable media include memory 320 and/or storage 340. As should be readily understood, the term computer-readable storage media includes the media for data storage such as memory 320 and storage 340, and not transmission media such as modulated data signals.

Any of the disclosed methods can be implemented using computer-executable instructions stored on one or more computer-readable media (e.g., non-transitory computer-readable media, such as one or more optical media discs, volatile memory components (such as DRAM or SRAM), or nonvolatile memory components (such as flash drives or hard drives)) and executed on a computer (e.g., any commercially available computer, proprietary computer, purpose-built computer, or supercomputer, including smart phones or other mobile devices that include computing hardware). Any of the computer-executable instructions for implementing the disclosed techniques, as well as any data created and used during implementation of the disclosed embodiments, can be stored on one or more computer-readable media (e.g., non-transitory computer-readable media). The computer-executable instructions can be part of, for example, a dedicated software application, or a software application that is accessed or downloaded via a web browser or other software application (such as a remote computing application). Such software can be executed, for example, on a single local computer (e.g., as a process executing on any suitable commercially available computer) or in a network environment (e.g., via the Internet, a wide-area network, a local-area network, a client-server network (such as a cloud computing network), or other such network) using one or more network computers.

For clarity, only certain selected aspects of the software-based implementations are described. Other details that are well known in the art are omitted. For example, it should be understood that the disclosed technology is not limited to any specific computer language or program. For instance, the disclosed technology can be implemented by software written in C, C++, Clojure, Common Lisp, Dylan, Erlang, Fortran, Go, Haskell, Java, Julia, Python, R, Scala, Scheme, SQL, XML, or any other suitable programming language. Likewise, the disclosed technology is not limited to any particular computer or type of hardware. Certain details of suitable computers and hardware are well-known and need not be set forth in detail in this disclosure. Furthermore, any of the software-based embodiments (comprising, for example, computer-executable instructions for causing a computer to perform any of the disclosed methods) can be uploaded, downloaded, or remotely accessed through a suitable communication means. Such suitable communication means include, for example, the Internet, the World Wide Web, an intranet, software applications, cable (including fiber optic cable), magnetic communications, electromagnetic communications (including RF, microwave, and infrared communications), electronic communications, or other such communication means.

### IV. EXAMPLES

### Example 1

### Thermal Analysis in Complex Fluids

### Materials and Methods

Chemicals and Reagents: Standard E. coli lysate was purchased from Bio-Rad (Hercules, Calif.) and received as a lyophilized powder. E. coli lysate samples were prepared by resuspending the appropriate mass of powder in standard PBS Buffer. Standard buffer for all experiments contained 150 mM NaCl, 10 mM potassium phosphate, 15 mM sodium citrate adjusted to pH=7.4 with hydrochloric acid. Samples of the HEK293 cellular supernatant, Lot number: 03U27020D; unpurified angiotensin converting enzyme 2 (ACE2) expressed in human embryonic kidney cells (HEK293) received in the preparation media Lot number: 02U2802LL; isolated, purified ACE2 Lot number: 04U27020GC; and purified receptor binding domain (RBD) from SARS-CoV-2, Lot Number:05U22020TWB were purchased from RayBiotech (Peachtree Corners, Ga.). All HEK293 solutions had a volume of 200 Captopril and Lisinopril were purchased from Sigma Aldrich (St. Louis, Mo.). Streptavidin was from Alfa Aesar (Haverhill, Calif.); biotin from VWR (Radnor, Pa.).

Preparation of Samples for DSC Measurements: E. coli lysate was prepared by resuspending powdered lysate in standard PBS buffer to make a 2.7 mg/mL lysate solution. The HEK923 cell lysate was the supernatant after initial centrifugation of the crude cell lysate and received in liquid form at unknown concentration. Working solutions had a total volume of 500 μL containing 400 μL of HEK293 lysate and 100 μL standard buffer. Streptavidin, captopril, lisinopril, and biotin were prepared in standard buffer. All samples were incubated for 24 hours at 4° C.

DSC Measurements: DSC melting experiments were made using a CSC differential scanning microcalorimeter (now T.A. instruments, New Castle, Del.). Samples were prepared by adding specific reaction components to pre-prepared media. For DSC melting experiments, the sample heating rate was approximately 1° C./min while monitoring changes in the excess heat (microwatts) of the sample versus temperature [6-8]. In their primary form, melting curves or thermograms are displayed as plots of changes in microwatts measured for the sample, versus temperature. Since all experiments involved investigating interactions of ligands with proteins using DSC, we were interested in testing the sensitivity or our approach to detect proteins and protein/ligand interactions in the background solution comprised of the crude cell lysate. Consequently, all DSC measurements were made on samples in the lysate media, E. coli lysate or HEK923 supernatant, where samples were prepared. Thermograms were acquired for the complex media background, several proteins, and mixtures of these proteins with ligands, i.e. drugs or other another protein.

Data Reduction and Analysis: Where the protein concentration was known, further refinement and normalization of the data provided the familiar DSC melting curve in the form of a plot of excess heat capacity, ΔC_{P}, of the sample as a function of increasing temperature. Plots of ΔC_{P} versus temperature are commonly termed DSC thermograms. In this case, the enthalpy of the transition can be estimated from the integrated area under the thermogram. These enthalpies can be used to gain valuable insight into protein structural stability (Eskew et al., Anal Biochem 2021, 612:113843). If the precise mass (concentration) of expressed protein was not known, thermograms were alternatively displayed in more primary form as plots of microwatts (μW) versus temperature. For these measurements, meaningful transition enthalpies cannot be determined.

Baselines of the raw ΔC_{P} or μW versus temperature thermograms for E. coli were determined using a three point polynomial fit, over the temperature range of the transition, which produced a nearly straight line from 40° C. to 110° C. Baselines of the raw ΔC_{P} or μW versus temperature thermograms for HEK293 samples were determined by connecting a straight line between data points at 40° C. and 100° C. This baseline was then subtracted from the raw curves, producing baseline corrected thermograms used for further comparisons and analysis. Between initial and final temperatures, ΔCₚ, for proteins is known to vary from 7-10 kcal/mol·K (Watanabe et al., Pharm Res. 2001, 18:1775-1781; Shrake et al., Vox sanguinis 1984, 47:7-18). Baseline choices served to effectively remove this difference (Eskew et al., Anal Biochem 2021, 612:113843; Lang et al., Biotechnology Progress 2015, 31:62-69). The baseline-subtracted thermograms are displayed. For all experiments thermograms of the media alone served as the background that was subtracted from thermograms of samples containing drugs and/or proteins in the same media. These difference thermograms provided enhanced visualization of sample-specific changes, corresponding predominantly to the ligand/protein interactions of concern.

### Results

Overview: Investigations were aimed at defining thermograms of the media background, and determining whether thermogram measurements have sufficient sensitivity to detect the presence of protein alone in media, and effects of ligand binding on the protein against the background of complex media. Results are reported for several examples conducted under different conditions.

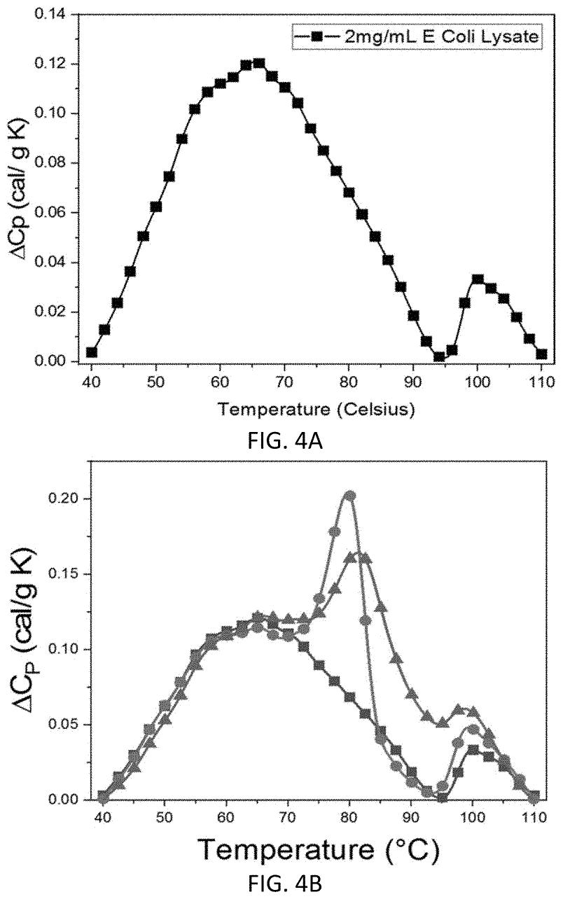

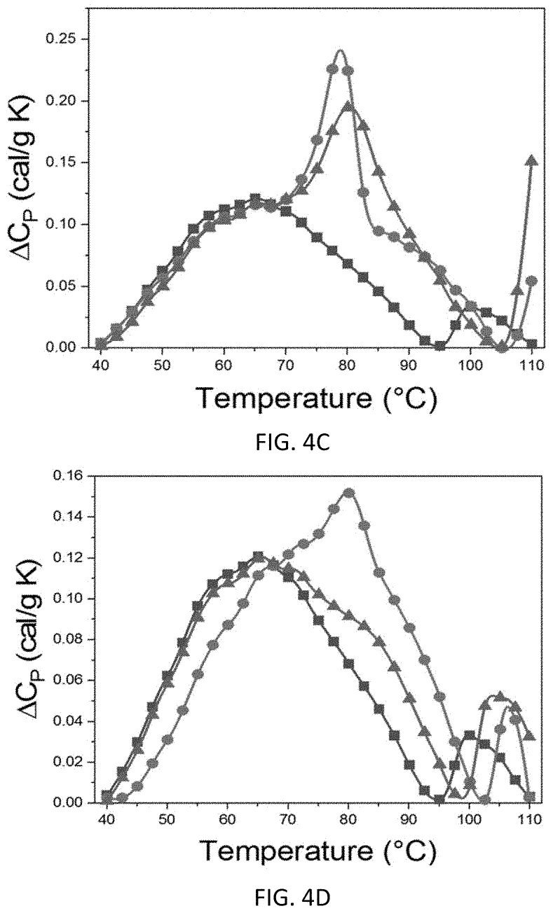

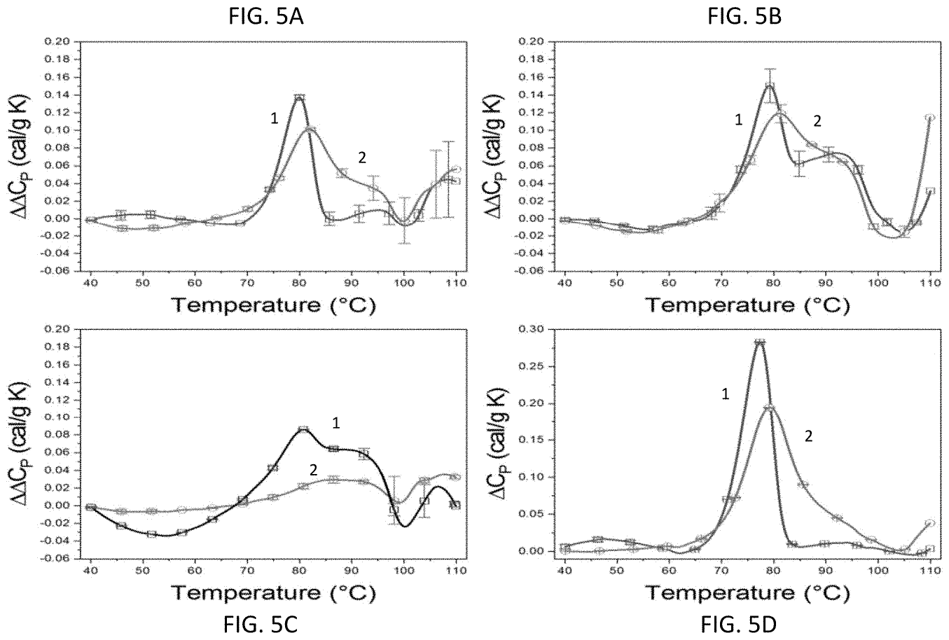

Streptavidin-Biotin: Initial investigations explored streptavidin-biotin binding in reconstituted E. coli lysate. Binding behavior was examined under several conditions meant to mimic what might be encountered in an actual expression system. FIGS. 4A-4D and 5A-5D present results of this initial study. The thermogram of the E. coli lysate alone served as the background against which streptavidin-biotin binding was examined. Along with that of the media (alone) thermograms for lysate solutions containing streptavidin in different amounts, and streptavidin plus two molar equivalents of biotin in the background media solution are also shown in FIGS. 4A-4D. For each condition at least two independent experiments were conducted. Error bars shown in FIGS. 5A-5D indicate the experimental reproducibility, which is quite good.

Thermograms for streptavidin alone and the streptavidin/biotin mixture as shown in FIGS. 4B-4D were measured under three different conditions. Condition-I was meant to mimic a 33% protein expression system. That is, the solutions contained 1 mg/mL of streptavidin added to 2 mg/mL of E. coli lysate. The streptavidin/biotin mixture contained streptavidin plus two molar equivalents of biotin in 2 mg/mL of E. coli lysate. Under Condition-I streptavidin concentration was half that of the lysate concentration making the total streptavidin concentration approximately 33% (w/w %). The three thermograms shown in FIG. 4B correspond to Condition-I.

The 33% protein expression system was further investigated with half the concentrations of the components in Condition-I. For Condition-II samples contained 0.5 mg/mL of streptavidin added to 1 mg/mL of E. coli lysate; and 0.5 mg/mL streptavidin plus two molar equivalents of biotin in 1 mg/mL of E. coli lysate. Results for Condition-II are shown in FIG. 4C. Even at half the concentrations of Condition-I, Condition-II displayed comparable signal resolution.

Examples thus far have demonstrated clear detection of ligand binding in a crude lysate at a 33% (w/w %) of total protein in the media (under Conditions-I and -II). While this value is not outside the range of possibilities, ˜10% (w/w %) is likely a more realistic moderate level of protein expression (Schellekens, Nephrology Dialysis Transplantation 2005, 20:iv31-iv36). To simulate this concentration range 0.25 mg/mL of streptavidin was added to 2 mg/mL lysate, corresponding to ˜11% expression (Condition-III). Results are shown in FIG. 4D. Comparison of the thermograms in FIGS. 4B-4D clearly shows effects of biotin binding on the streptavidin thermogram, and sensitivity of the measurement to concentration. There are differences in FIGS. 4B-4D, but the trends of the thermograms and shifts with ligand binding are consistent under each of the three conditions. Under these conditions, different signals from streptavidin alone and streptavidin/biotin complexes in the lysate background are clearly detectable. While very good reproducibility was found between subsequent experiments over the range of 40-90° C., results collected above ˜100° C. displayed more variability probably due to slight aggregation in the lysate at high temperatures. Consistent with this supposition, variability over this range was more pronounced for samples containing 2 mg/mL lysate (FIGS. 4B and 4D) than for 1 mg/mL lysate (FIG. 4C), as expected.

To enhance thermogram features of streptavidin and streptavidin/biotin complexes and their differences, difference thermograms where obtained by subtracting the thermogram for E. coli lysate alone (background, FIG. 4A) from the thermograms of streptavidin or streptavidin plus biotin. Results for Conditions I-III are shown in FIGS. 5A-5D. It would be expected that difference thermograms for Conditions-I and II, FIGS. 5A and 5B respectively, would be identical since thermograms are normalized by weight where ΔC_{P} is defined by cal/g K. However, there were slight differences between FIGS. 5A and 5B that warrant remark. The streptavidin peaks in FIGS. 5A and 5B display roughly equal peak heights at ˜79° C., also the addition of biotin causes a similar ˜1° C. shift in Tm for the dominant peak, with a corresponding decrease in overall peak height. The primary difference occurs around the secondary peak that arises for streptavidin, in FIG. 5B, at ˜95° C. This secondary peak was present and reproducible in all experiments under Condition-II. The source of this behavior is likely the lowered signal to noise ratio resulting from conversion of μW to ΔC_{P} under Condition-II; where small deviations above the background are magnified when accounting for the lesser mass of total protein in FIG. 5B. That is, any deviations in FIG. 5B would be magnified by 100% compared to those in FIG. 5A because of the difference in total protein mass of the solution (3 mg/mL in FIGS. 5A and 5C versus 1.5 mg/mL in FIG. 5B).

Difference plots determined at the relatively lower concentrations of Condition-III are displayed in FIG. 5C. Clearly, the plots at 11% protein under Condition-III (FIG. 5C) are somewhat different than those obtained at the higher 33% protein concentration (Conditions-I and —II, FIGS. 5A, 5B). Even so, in analogy to results for the 33% examples in FIGS. 5A and 5B, under Condition-III at 11% streptavidin, the temperature of the predominant peak (at ˜80° C.) is preserved but decreases in intensity at the lower concentration; while the peak at ˜110° C. increases. For reference, thermograms of streptavidin and streptavidin plus biotin alone in buffer (not lysate) are shown in FIG. 5D. These curves serve as the control for the isolated effect of streptavidin/biotin binding. FIG. 5D shows binding is typified by a temperature shift of 1.8° C., 20% reduction in peak height and increased width of the thermogram. Observations in FIG. 5D are fully consistent with published calorimetric measurements of thermal denaturation of streptavidin (González, J of Biol. Chem. 1997, 272:12888-11294). Without biotin streptavidin has a melting temperature around 80° C. and shifts up from 1-2° C. under Conditions I-III.

Under all three conditions the shift of the thermogram of streptavidin in the presence of biotin was comparable. The 20% reduction in peak height for streptavidin-biotin alone, was consistent with those observed for streptavidin-biotin under Conditions I-III. The temperature shifts of streptavidin+biotin in buffer and under Conditions I-III are summarized in Table 1. The shift for streptavidin and streptavidin plus biotin in buffer is 1.8° C. This shift decreases to 1.4 and 0.9° C. in E. coli lysate at decreased concentrations. Since this shift is the primary metric for ascertaining the presence of binding; the comparable temperature shifts summarized in Table 1 demonstrate the detection capabilities of the measurements, even at relatively lower concentrations.

TABLE 1
Temperature shift for Streptavidin + Biotin Thermograms
for Conditions I-III

| Sample | Streptavidin | Streptavidin + | Δ Tm |
|---|---|---|---|
| | Tm (° C.) | Biotin Tm (° C.) | (° C.) |
| 1 mg/mL Streptavidin | 77.39 ± 0.05 | 79.17 ± 0.06 | 1.79 ± 0.02 |
| (control) | | | |
| 2 mg/mL Streptavidin | 79.70 ± 0.02 | 81.16 ± 0.07 | 1.46 ± 0.05 |
| (33%, Condition-I) | | | |
| 1 mg/mL Streptavidin | 79.03 ± 0.18 | 79.96 ± 0.36 | 0.93 ± 0.18 |
| (33%, Condition-II) | | | |
| 2 mg/mL Streptavidin | 79.82 ± 0.10 | 80.71 ± 0.12 | 0.89 ± 0.02 |
| (11%, Condition-Ill) | | | |

The results with the streptavidin-biotin system clearly demonstrate sensitivity of the DSC measurements to detect presence of streptavidin and biotin binding in the E. coli lysate; the directed primary purpose of the study. Differences between the thermograms for streptavidin alone, and streptavidin plus biotin in E. coli lysate were clearly discernable. Finding that binding of a ligand to a target protein in a crude lysate can be detected by DSC was a compelling preliminary result, and we were motivated by these results to venture further. However, the streptavidin-biotin system represents an ideal, and probably unrealistic, model of an expression system. Unrealistic because in the streptavidin-biotin system the total protein mass and mass percentage are precisely known prior to the experiment. Also, even in our moderate expression example (˜11% w/w %) may still be higher than some or most real expression systems. In a real expression system, the actual amount of desired protein mass and percentage may not be known, or easily determined, particularly in early stages of development. In the next examples, products of a real expression system were used where concentrations of the expressed protein and lysate were unknown.

Angiotensin Converting Enzyme (ACE2): ACE2 expressed in liquid culture of human kidney cells (HEK293) was purchased from a commercial vendor. The supernatant containing ACE2 was provided from the vendor along with standard supernatant without expressed protein. The supernatant served as the background solution. Protein mass of lysate supernatant was unknown. Protein samples were provided in a specified volume, but the mass of ACE2 in that volume was also not known. Consequently, the actual mass of ACE2 in the sample solution, or what fraction of the total protein mass in the supernatant was comprised of ACE2, were unknown. This low level of information regarding the expressed protein in the supernatant likely resembles the situation that could be encountered in early-stage assessment of biologics expressed in vitro.

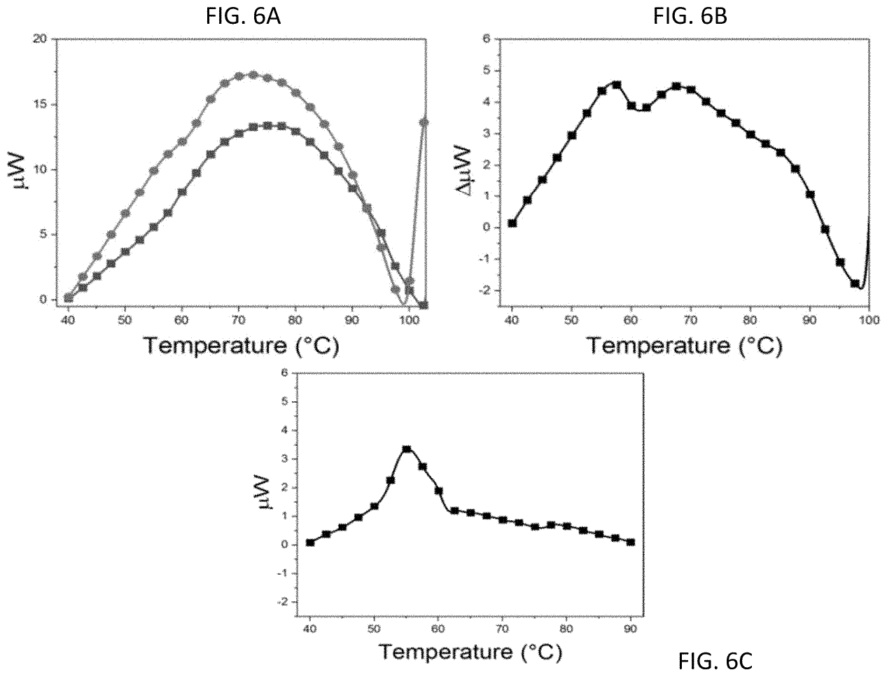

The thermogram measured for the supernatant, without ACE2 present, served as the background against which comparisons were made. Results are shown in FIGS. 6A-6C. FIG. 6A displays the thermograms for the supernatant background and a solution of the supernatant containing an unknown amount of expressed ACE2. These curves have similar shapes but there is a clear difference for the thermogram of the supernatant containing ACE2. To isolate and enhance the specific contributions to the thermogram of ACE2 protein, the background thermogram for the supernatant alone was subtracted from that for ACE2 in the supernatant. The difference thermogram corresponding to ACE2 protein alone is displayed in FIG. 6B; and shows two peaks at 55.6° C. and 70.6° C. This observation is in precise agreement with published results of DSC measurements on unmodified ACE2, that also found two peaks at precisely the same temperatures (Voronov et al., FEBS Letters 2002, 522:77-82). The peak at ˜55° C. was assigned to correspond to the C-domain of the protein; while the peak at ˜70° C. was assigned to the N-domain of ACE2 (Id.).

For comparison purposes, purified samples of isolated ACE2 were purchased and DSC thermograms were measured for those samples. The thermogram for the purified ACE2 that was purchased is shown in FIG. 6C. When compared with the thermogram for unpurified ACE2 in FIG. 6B, there are clear differences. Most notedly, the thermogram for purified ACE2 in FIG. 6C does not match the thermograms for unpurified ACE2 in FIG. 6B; nor does it match the DSC thermograms reported for somatic bovine ACE2 (Id.). These differences between the measured thermograms for purified ACE2, and those measured for unpurified ACE2 and purified ACE2 reported in the literature (Id.) are remarkable and suggest the purification process must have fundamentally affected the structure of ACE2.

To note, the purchased unpurified and purified ACE2, purportedly contain only the extracellular domain. Interestingly modeling of ACE2 secondary structure from amino acid composition demonstrated that the N and C domains responsible for the dual thermogram peaks corresponded to the sequence from residues 1-734 (Id.). In comparison, the expressed region for the purchased ACE2 was from Gln18-Ser740. Thus, this region should also have included both domains. It is possible that observed differences are due to the different origins of the ACE2 samples, bovine versus human. However remote, this possibility does not explain the presence of the extra peak at ˜70° C. in the expressed unpurified human ACE2.

These observations prompt several questions. If expression of Gln18-Ser740 is sufficient to suppress formation of the N domain peak at ˜70° C., what are the possible origins of this peak on the thermogram of unpurified ACE2? The purification process could have affected the structure of ACE2, consistent with diminishment of the peak at ˜70° C. on the thermogram of the purified protein. That suggests the structure of the N-domain of the protein was somehow affected by purification. Also, if the serum-free cell supernatant of the unpurified ACE2 contains remnants of cellular ACE2 from the expression system, could these be responsible for the peak at ˜70° C.? This seems unlikely since the sizes of the two peaks on the thermogram of unpurified ACE2 are essentially equivalent. If the cellular ACE2 exhibited normal dual peak behavior as reported in reference 10, in FIG. 6B we would have expected to see a larger peak at ˜55° C. due to excess purified ACE2. But this was not observed.

Alternatively, the possibility exists that components in the unpurified sample bind to ACE2 thereby causing the observed dual peaks in the thermogram. This is viewed as unlikely. Previous results on ligand binding to proteins demonstrated that ligand binding is almost universally linked to changes of Tm on measured thermograms (Eskew et al., Anal Biochem 2021, 612:113843; Eskew et al., Anal Biochem 2021, 628:114293). Such Tm shifts associated with ligand binding are a well-known manifestation of Le Chatelier's principle (Id.; Celej et al., Biochimica et Biophysica Acta-Proteins and Proteomics 2005, 1750:122-133; Dassie et al., J Chem Ed 2005, 82:85). Since Tm for the two observed thermogram peaks are essentially identical to those reported (Voronov et al., FEBS Letters 2002, 522:77-82), ligand binding to ACE2 seems an unlikely explanation for the ˜70° C. peak. Additionally, ACE2 samples were purified using His-tag chromatography. If a ligand in the unpurified sample were to bind strongly enough to shift some portion of the ACE2 thermogram from ˜55° C. to ˜70° C., some ligand would have been expected to remain bound and survive the chromatography purification step. As shown in FIG. 6C for the purified sample, this is not the case.

Conventional methods for protein purification such as gel chromatography purify proteins based on their mass. Consequently, measurements of protein mass by these methods are accurate so long as the mass does not change during the purification process. Such measurements might be expected to be largely insensitive to changes in structural integrity of the protein, possibly brought about by different steps in the purification process. Conversely, as evidenced by the thermogram in FIGS. 6A-6C, even though the mass of the protein was unchanged, structural integrity was apparently disrupted. Implications of this observation are highly significant for the following reason. Biologics, proteins or protein complexes, are intrinsically designed to target specific receptors. Much of the target specificity derives from critical interactions dictated by the overall structure in solution adopted by the biologic. Supposing that purification steps substantially alter the biologic structure, activity of the compound could be adversely affected; and the biologic could display diminished efficacy.

For example, consider the following scenarios. Suppose the drug compound was designed to specifically bind the N-domain of ACE2, or expressed and purified ACE2 was used to test binding of drug compounds. If the N-domain of the ACE2 used in the binding assay was disrupted the binding activity could be considerably affected and provide a false negative result. Such a result in the context of screening could inadvertently lead to elimination of potentially active compounds. Additionally, if the N-domain were present but with a substantially altered structure (from truncated expression or purification), it is possible that the altered structure could actually have a higher binding affinity than the native N-domain structure. Again, leading to anomalous results, in this case, a false positive. Although these are hypothetical scenarios, they highlight the unique utility of thermogram analysis and emphasize valuable insights in the screening processes prior to protein purification. The aforementioned hypothetical scenario is especially relevant considering the numerous recently reported studies of SARS-CoV-2 (COVID-19); where the receptor binding domain (RBD) of COVID-19 specifically targeted ACE2 (Zhang et al., Intensive Care Medicine 2020, 1-5; Prabakaran et al., Biochem and Biophys Res Comm 2004, 314:235-241; Khelfaoui et al., J of Biomolecular Structure and Dynamics 2020, 1-17; Smith et al., DOI 10.26434/chemrxiv.11871402.v4). In which case, damaged ACE2 used to assay binding could provide spurious results.

Binding of inhibitors to unpurified ACE2: Ligand binding measurements were also conducted on unpurified ACE2 in HEK293 cell supernatant plus added ligands. This next example involved a series of trials aimed at detection of binding of known ligands to ACE2 in expressed, unpurified form, in the complex media background. Specifically, the ligands were captopril and lisinopril, both clinically active as ACE2 inhibitors to treat high blood pressure and heart failure. Thermograms were measured for solutions of ACE2 expressed in an unknown amount, plus captopril or lisinopril added to the supernatant.

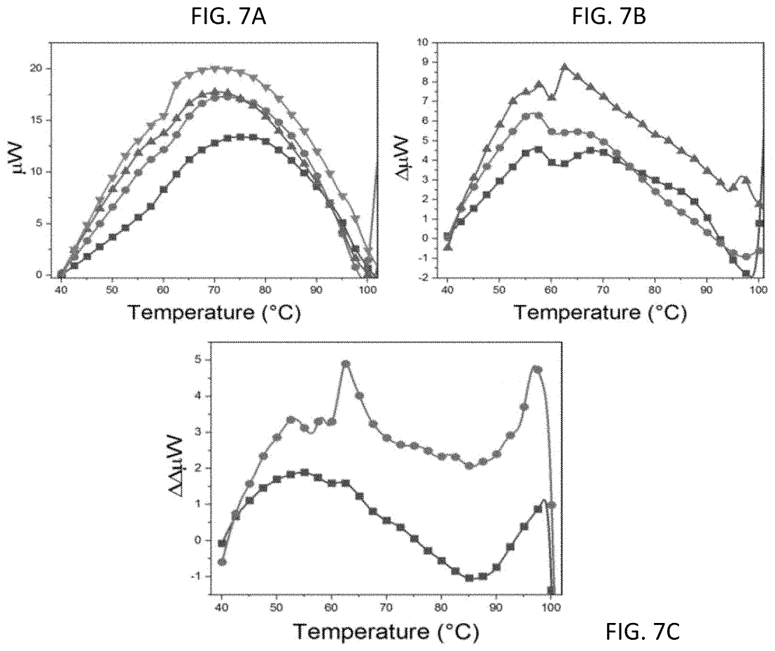

Results are summarized in FIGS. 7A-7C. Thermograms of the lysate supernatant background, expressed ACE2 in the supernatant background, and the same solution plus captopril or lisinopril (at 250 μM) are shown in FIG. 7A. In the media background all four curves have similar shapes, but also display clear differences. To better emphasize these differences, the thermogram of the background lysate was subtracted from the other three thermograms in FIG. 7A rendering the difference curves shown in FIG. 7B. These curves for ACE2 alone and ACE2+captropril and ACE2+lisinopril are clearly different, but there are some similar features. To further isolate and emphasize effects of the drugs alone on ACE2 stability, the background-subtracted thermogram for ACE2 alone in FIG. 7B was subtracted from the background-subtracted thermogram of the mixture of ACE2+captropril and ACE2+lisinopril (also FIG. 7B). These difference curves are shown in FIG. 7C. By removing contributions to the thermogram of both the supernatant background, and expressed ACE2, changes to ACE2 strictly due to effects of the ligands (captopril or lisinopril) on the ACE2+ligand thermogram emerge in FIG. 7C. These isolated curves for lisinopril and captopril displayed in FIG. 7C reveal remarkable differences between the two drugs. Lisinopril seems to have the largest effect on the region centered at 55° C. corresponding primarily to the C-domain region of the ACE2 thermogram; suggesting this may be the primary region of interaction between the drug and ACE2. As seen in FIG. 7C, the difference thermogram of ACE2 containing captopril shows a relatively lesser effect on ACE2 compared to lisinopril. The difference thermogram for lisinopril suggests that, unlike captopril, lisinopril affects the stability of both the C and N domains of ACE2. These results for ACE2 and the ligands highlight different binding interactions for the two ligands that belong to the same class of drugs. Lisinopril is the stronger binder and has a greater effect on ACE2 activity than captopril. This observation is consistent with previous molecular docking studies that showed lisinopril is a stronger binder to ACE2 and has a higher affinity for the N-domain than captopril (Khelfaoui et al., J of Biomolecular Structure and Dynamics 2020, 1-17; Hijkata et al., FEBS Letters 2020, 594:1960-1973; Guy et al., Biochemistry 2003, 42:13185-13192).

Interactions of COVID-19 RBD with ACE2: Examples described thus far involved analysis of systems meant to mimic small molecule drug ligand binding to expressed proteins. Experiments in the next example were designed to ascertain effects of protein-protein interactions with unpurified expressed protein in complex media. Such as may be the case when screening biologics which might be comprised of peptides or proteins that bind a particular expressed protein; or screening of potential protein targets for a specific protein ligand. A number of studies have determined the primary route of cellular infection by COVID-19 is binding of the viral RBD to ACE2 (He et al., J of Medical Virology 2020, https://doi.org/10.1002/hmv.25766; Zhang et al., Intensive Care Medicine 2020, 1-5; Yan et al., Science 2020, 367:1444-1448).

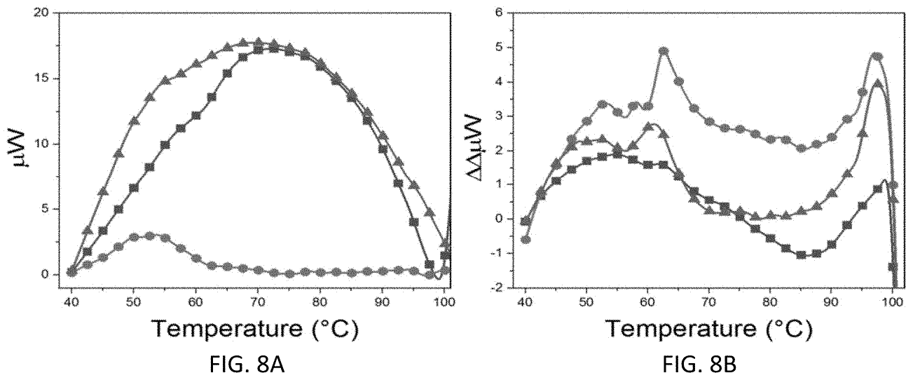

For this example, interactions of the receptor-binding domain (RBD) of COVID-19 with ACE2 were investigated. In this case, the RBD of COVID-19 was the ligand. Results are shown in FIGS. 8A-8B. Thermograms of purified RBD alone, unpurified ACE2 in the supernatant background and the purified RBD from COVID-19 added to unpurified ACE2 in the cell supernatant, are shown in FIG. 8A. To better visualize contributions from RBD binding to unpurified ACE2 in the complex media supernatant, the thermograms of unpurified ACE2 in the supernatant and RBD alone in buffer were subtracted from the composite thermogram measured for the mixture of unpurified ACE2 and RBD. The resulting difference thermogram for the effects of the RBD protein ligand on unpurified ACE2 are displayed in FIG. 8B (▴). The largest intensity occurs between 50 and 60° C. suggesting that that the interaction of RBD with ACE2 is primarily with the C-domain whose position shifts with binding.

COVID-19, captopril and lisinopril interactions with ACE2: Since it has been determined that the route of COVID-19 viral infection is through binding of the RBD on the virus coat to the ACE2 protein, computational studies have been conducted for the purpose of designing or identifying drugs that can disrupt the ACE2/RBD interaction (Smith et al., ChemRxiv. 2020; Dong et al., Drug Discoveries & Therapeutics 2020, 14:58-60; Jin et al., bioRxiv 2020; Liu et al., ACS Publications 2020). In alternative approaches, researchers have also employed computational tools to virtually screen approved drugs for their potential binding to ACE2, and the RBD protein. Through this approach at least 40 commercially available compounds with suspected binding activity for ACE2 and RBD have been identified; offering them as possible inhibitors for the virus ((Smith et al., ChemRxiv. 2020; Dong et al., Drug Discoveries & Therapeutics 2020). However, experiments have been slow to confirm these computational predictions which currently amount to little more than theoretical conjecture. Confirmation has been slow mostly due to costs associated with testing compounds against the relatively expensive RBD and ACE2 protein samples.

The background subtracted thermogram for mixtures of ACE2 and RBD is shown in FIG. 8B along with the difference curves for captopril and lisinopril. These results qualitatively demonstrate that, in terms of their thermogram responses, the interaction of COVID-19 RBD with ACE2 more strongly resembles lisinopril than captopril binding of ACE2. This observation might indicate that lisinopril, by binding strongly and in a similar manner to ACE2, may be an effective inhibitor of RBD binding. While lisinopril has proven ineffective at inhibiting COVID-19 infection, primarily by causing an upregulation of ACE2 in the body and subsequently providing more opportunities for infection; molecular modeling studies showed that based on binding characteristics lisinopril would be effective at inhibiting RBD binding to ACE2 (Zhang et al., Intensive Care Medicine 2020, 1-5; Smith et al., ChemRxiv. 2020; Li et al., Science 2005, 309:1864-1868).

For ACE2, quantitative binding constants could not be accurately determined due to the unknown mass. With accurate protein masses, it is possible that the relative changes of thermogram integrated area (evaluated enthalpy) and Tₘ with increasing ligand concentration, can be used to measure ligand binding constants.

### DISCUSSION

The foregoing results demonstrate the two-fold application of DSC measurements for structural analysis of expressed proteins and rapid and sensitive detection of ligand binding of targeted proteins in complex culture media. This process provides a new means to probe the protein interactome and analyze complex solutions containing biologics produced by in vitro expression systems. For specific examples presented here attention focused on protein/small drug molecule interactions, but the process can be broadly expanded to interrogate protein-protein, antibody-antigen, protein-peptide, or interactions between any combination of biomolecules and small drug molecules.

Contrast this with conventional drug screening technologies (isothermal titration calorimetry, surface plasmon resonance, mass spectrometry, etc.) that require the use of highly pure compounds and solvents. Given the extreme cost and time associated with sample purification the ability to characterize “impure” solutions and assess protein binding interactions provides a novel and powerful alternative for initial screening of new biologic compounds produced by in vitro expression. Results convincingly demonstrated detection of binding of small molecule drug compounds to proteins in unpurified lysate media. In a comparative manner, the process was able to detect ligand binding despite no precise knowledge of the protein composition of the HEK293 lysate e.g. total mass of protein in solution or the percentage of expressed protein. Applications for the capability of detecting binding in unpurified lysates does are not limited to screening of biologic compounds. Based on results for detection of RBD binding from COVID-19 to ACE2 the analytical process provides a compelling application in the detection and analysis of viral outbreaks.

Unique thermograms can also serve as the basis for screening biofluids such as plasma, mucus, cerebrospinal fluid, sputum, urine, etc.) for the presence of live virus. Screening of the different biofluids combined with the analysis of expressed proteins in complex media enables a means for rapid screening of existing compounds for their efficacy in inhibiting binding to either protein targets on the virus, or the whole virus itself.

Other than culture and amplification (both costly and time intensive), there currently are no methods available for accurate and rapid screening of biofluids for the presence of active virus. In this application, the DSC approach is positioned to perhaps make significant contributions. With the presence of virus particles in diseased biofluids comes the expectation that thermograms of infected samples will be significantly different compared to the normal thermogram for that biofluid. In this way thermograms can be extremely sensitive indicators of viral infection.

While plasma is not the primary tissue compartment for COVID-19, it is the primary compartment for other infectious diseases such as human papillomavirus and hepatitis (Chan et al., J of Clinical Microbiology 2020, 58(5); Lescure et al., The Lancet Infectious Diseases 2020, 20(6):697-706; Zhang et al., Transfusion 2016, 56:2248-2255; Suomela, Transfusion Medicine Reviews 1993, 7:42-57). The capture process can be easily adapted to any biofluid or protein target. In principle, the actual physical isolation of virus particles directly from plasma, using the capture reagent, can be a far more accurate indicator of active infection than detection of remnants of viral RNA using PCR.

Although the above example focused primarily on COVID-19, the approach is not limited to this virus. Embodiments of the disclosed method provide a universal format and complementary tool for analysis of virtually any disease (virus-based, or otherwise).

### Example 2

### Equivalence of Transition Heat Capacities of Proteins and DNA

### Materials and Methods

Chemicals and reagents: Standard buffer for all experiments contained 150 mM NaCl, 10 mM potassium phosphate, 15 mM sodium citrate adjusted to pH=7.4 with hydrochloric acid. Pure Proteins: Human Serum Albumin (HSA) (≥99% pure, Lot number: SLBT8667) and lysozyme (recombinant, expressed in rice, Lot number: SLCH2681). The above proteins were prepared in standard buffer and stored at 4° C. for at least 24 hours before use. Concentrations of HSA and lysozyme were confirmed spectrophotometrically at A₂₈₀.

DNA: The DNA was a 20-base pair hairpin purchased from IDT and received following a standard desalting routine. The DNA standard sequence is 5′-CGG GCG CGT TTT CGC GCC CG-3′ (SEQ ID NO: 6). Lyophilized DNA was resuspended in standard buffer and stored at 4° C. DNA concentration matched the manufacturer specification as determined spectrophotometrically at A₂₆₀.

DSC Measurements: DSC melting experiments were made using a CSC differential scanning microcalorimeter (now T.A. instruments, New Castle, Del.). For DSC melting experiments, the sample heating rate was approximately 1° C./min while monitoring changes in the excess heat (microcalories) of the sample versus temperature. Sample volumes for DSC melting experiments were 0.5 mL.

Data Reduction and Analysis: Thermograms of proteins were displayed in primary form as plots of microcalories (μcal) versus temperature. Baselines of the raw ΔCₚ or μcal versus temperature thermograms were determined using a four-point polynomial fit, over the temperature range of the transition, consistent with previous methods (Eskew et al., Anal Biochem 2021, 612:113843; Koslen et al., Adv in Biol Chem 2019, 9; Eskew et al., Anal Biochem 2021, 628:114293). For all protein mixtures the buffer baseline was then subtracted from the raw curves, producing baseline corrected thermograms used for further comparisons and analysis. Linear fits of peak height versus protein/DNA concentration were performed in triplicate and fit using Origin (Pro), Version 2021b, Origin Corporation, Northampton, Mass.

### Results and Discussion

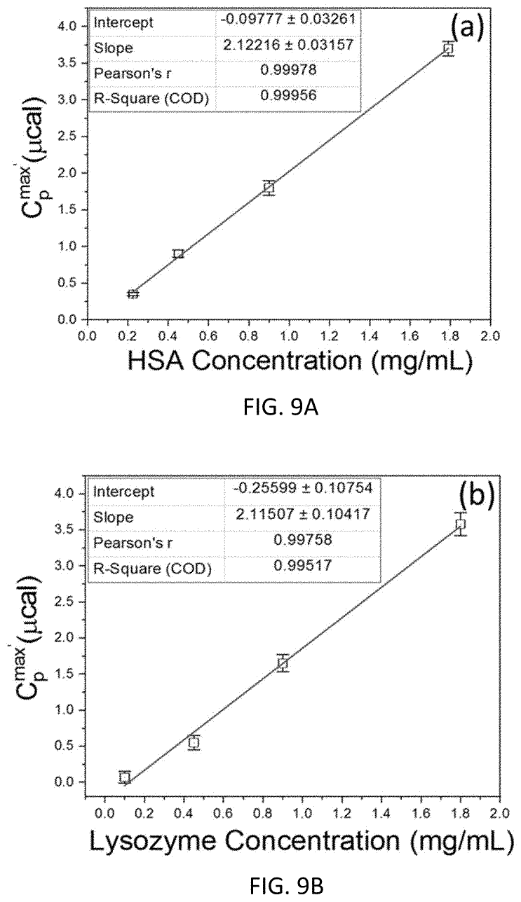

To demonstrate the equivalence of transition heat capacity for proteins, human serum albumin (HSA) and lysozyme were used as examples of typical globular proteins. To determine transition heat capacities, C_{p,p}, for the individual proteins, as a function of mass, thermograms were measured for titrations of HSA and lysozyme. C_{p,p} was determined using equation (4) supra. In FIGS. 9A and 9B, calorimetric peak height (absolute heat capacity, Cₚ^{max′}, is plotted versus protein concentration (mg/mL). According to equation (4) the slope of the resulting plot equals the transition heat capacity minus the PSV (Slope=C_{p,p}−PSVₚ). Rearrangement of this equation provides C_{p,p}, i.e.:

C_{p,p}=Slope+PSVₚ (6)

Four concentrations for HSA and lysozyme were used to determine C_{p,p}. Three measurements were made for each protein solution (FIGS. 15A, 15B). Since Tₘ should be independent of the protein concentration, as expected, across the measured concentration range the Tₘ remains unchanged. Measured Tₘ values for HSA and lysozyme were T_{m,HSA}=63.62±0.11° C. and T_{m,Lys}=64.77±0.16° C.

PSV values for the proteins taken from the literature were 0.733 mL/mg for HSA and 0.703 mL/mg for lysozyme (Gomez et al., Proteins: Structure, Function, and Bioinformatics 1995, 22:404-412; Kholodenko et al., Anal Biochem 1999, 270:336-338; Eskew et al., Anal Biochem 2021, 612:113843; Peters Jr, T., All about albumin: biochemistry, genetics, and medical applications, Academic press 1995; Eskew et al., Anal Biochem 2021, 628:114293). The observed transition heat capacity values for HSA and lysozyme were evaluated, from equation (6), C_{p,HSA}=2.86±0.03 mcal·g⁻¹·K⁻¹, C_{p,Lys}=2.82±0.13 mcal·g⁻¹·K⁻¹. Lysozyme results showed a slightly lower overall transition heat capacity and larger variance compared to HSA. Although of little concern, this small difference was likely due to the poor shelf stability of lysozyme solutions, which required preparation of multiple stock solutions and more precise timing of measurements. Even with these factors effecting the lysozyme samples, nearly exact C_{p,p} values were obtained, supporting the assertation that, normalized per mass, the transition heat capacity is essentially the same for these globular proteins.

The DNA was a 20-base single strand oligomer designed to form an intramolecular stem-loop “hairpin” structure; with eight base pairs in the duplex stem and four-base single strand loop on one end. The stability of the eight base pair sequence was designed to have a melting temperature above 90° C.; T_{m,DNA}=94.34±0.06° C. Again, as expected, this Tₘ was independent of DNA concentration, with no evidence of intermolecular duplex formation.

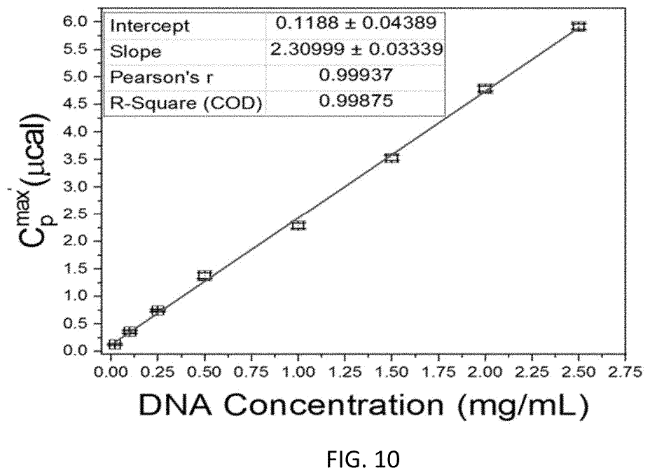

DSC thermograms were collected for DNA at concentrations from 0.02 mg/mL to 2.50 mg/mL. Experiments were performed in triplicate. Maximum peak heights on DSC thermograms, Cₚ^{max′} values, were determined for each DNA concentration, resulting in the curve shown in FIG. 10. The excellent linearity of the plot of Cₚ^{max′} versus DNA concentration (R²=0.999) confirms the linear dependence on mass for the DNA sample.

In an analogous manner to the proteins above, equation (6) was used to evaluate the observed transition heat capacity for the DNA hairpin. The literature value for short DNA oligomers PSV_{DNA}=0.55 mL/g, was employed (Le et al., Quadruplex Nucleic Acids 2012, 179-210). With this PSV, the transition heat capacity for the DNA hairpin was determined to be C_{p,DNA}=2.86±0.03 mcal·g⁻¹·K⁻¹.

Thus, C_{p,DNA} (2.86) is precisely the same as C_{p,HSA} (2.86) and nearly identical to C_{p,Lys} (2.82). Although only demonstrated for these three molecules, these results are consistent with “universal” values that have been evaluated for many proteins at 25° C. Although for only a single molecule examined here, the agreement of C_{p,DNA} with those of the proteins, suggests the potential equivalence for transition heat capacity values of proteins and DNA.

The above results can be used to evaluate the transition heat capacity of individual molecules, C_{p,DNA}=3.63×10⁻²⁰ mcal·molecule⁻¹·K⁻¹ for DNA and C_{p,HSA}=3.16×10⁻¹⁹ mcal·molecule⁻¹·K⁻¹ for HSA. This indicates that each HSA molecule has 8.7 times as much heat capacity as a single DNA molecule. Coincidently, the ratio of molecular weights of HSA to DNA is also 8.7. Thus, at a given mass in a sample, there will be 8.7 times more DNA molecules than HSA molecules. Likewise, for lysozyme C_{p,Lys}=6.79×10⁻²² mcal·molecule⁻¹·K⁻¹. Each lysozyme has 1.9 times as much heat capacity as a single DNA molecule, where again the ratio of molecular weights of lysozyme to DNA is also 1.9.

Examining the heat capacity per amino acid or base residue for each molecule, C_{p,HSA}=5.40×10⁻²² mcal·residue⁻¹·K⁻¹, C_{p,Lys}=5.26×10⁻²² mcal·residue⁻¹·K⁻¹ and C_{p,DNA}=1.81×10⁻²¹ mcal·base⁻¹·K⁻¹. Thus, the heat capacity of each DNA base is 3.36 and 3.45 times greater than an amino acid residue for HSA or lysozyme, respectively. Coincidently, this corresponds to a factor of 3.36 greater for the average molecular weight of each base of the DNA hairpin (382.1 g/mol) compared to each amino acid of HSA (113.7 g/mol). A similar factor 3.45 exists for the ratio of the molecular weight of each DNA base to lysozyme amino acid (110.0 g/mol). This observation suggests the heat capacity of proteins and DNA corresponds to the mass of individual bases or amino acid residues. Interestingly, this is entirely consistent with the proposition that molecular size is directly coupled to hydration enthalpy (Robinson et al., Biophys J 1999, 77:3311-3318; Privalov et al., European Biophysics J 2017, 46:203-224). Said another way, per residue, since DNA bases are approximately three times larger than amino acid residues their hydration enthalpies are likewise three times greater.

Observation of identical heat capacities for DNA and proteins, C_{p,p}=C_{p,DNA}, is intriguing and potentially implies a common origin for protein and DNA global structural stability due to hydration effects. While water and hydration effects are the most likely cause for this phenomenon, “hydration” is a simplistic explanation. As mentioned, water has multiple effects on protein stability from the native hydration shell to hydration of hydrophilic and hydrophobic groups. Within these effects there is likely enthalpy/entropy compensation that, per mass, is normalized for the specifics of small DNA oligos and large protein molecules. The exact nature and magnitude of hydration effects between large and small molecules is currently the subject of investigation.

There are practical applications for these results. For example, because of the equivalence of the transition heat capacity of proteins and DNA, the unknown mass of a protein can be quantitatively evaluated solely from the measured calorimetric peak height of the protein, when compared to the peak height for a known amount of DNA of the same mass.

It is noted that the transition heat capacity values we evaluated for lysozyme and HSA differ from those previously reported in the literature for the same proteins (Gomez et al., Proteins: Structure, Function, and Bioinformatics 1995, 22:404-412; Sirotkin et al., J Phys Chem B 2012, 116:4098-4105). The origin for this difference arises from use of the transition heat capacity at Tₘ compared to the native heat capacity evaluated at 25° C. reported in the literature. Nevertheless, within the same data group, i.e. same instrument, same baseline parameters, same data treatment, the data evaluated at Tₘ is internally consistent and again demonstrates the universal heat capacity for globular proteins at any temperature.

### Example 3

### Using DNA Standard Molecules

In preliminary experiments for the DNA hairpin, human serum albumin (HSA), and lysozyme, transition heat capacity values were evaluated for C_{p,DNA}=2.86±0.03 mcal·g⁻¹·K⁻¹, C_{p,HSA}=2.86±0.03 mcal·g⁻¹·K⁻¹, and C_{p,Lys}=2.82±0.13 mcal·g⁻¹·K⁻¹; showing negligible differences between them.

The stability of the six base pair hairpin sequence was chosen to have a melting temperature above 90° C.; actually 94.34±0.06° C. As expected, the measured Tₘ was independent of DNA concentration, with no evidence of intermolecular duplex formation. Plots of maximum peak heights on thermograms, Cₚ^{max′}, versus DNA concentration were used to construct a calibration curve. This was then used to calculate mass concentrations for unknown proteins in Table 2. Evaluated mass concentrations of proteins were in precise agreement for several different proteins, supporting the proposition that transition heat capacities are essentially the same for globular proteins.

TABLE 2
DNA Probe Mass Comparison

| | Peak | Evaluated | Independently |
|---|---|---|---|
| | Height | Mass | Determined |
| Sample | (μcal) | (mg/mL) | Mass (mg/mL) |
| HSA | 3.60 | 1.73 | 1.79 |
| HSA in plasma | 2.08 | 1.02 | 1.08 |
| ACE2 | 0.49 | 0.28 | 0.20 |
| Sars-CoV-2 RBD | 0.75 | 0.40 | 0.40 |
| E. coli lysate | 3.89 | 1.86 | 2.00 |
| ACE2 unpurified | (C) 0.09 | (C) 0.04 | — |
| | (N) 0.06 | (N) 0.03 | |
| Lysozyme | 3.55 | 1.69 | 1.80 |
| Streptavidin | 6.71 | 3.15 | 0.79 |
| 16mer DNA | 8.20 | 2.00 | 3.60 |

In preliminary experiments the initial DNA standard was employed to predict the mass concentrations of various proteins and complex fluids that were studied by DSC. Additionally a complementary DNA hairpin was designed with a melting temperature of about 70° C. that produced identical results. Results in Table 2 show for all cases (examined thus far), excellent agreement was obtained between masses determined using the DNA calibration curve and equation 5 supra (column 2) and masses determined by other independent means (column 3). For HSA in plasma, it was assumed that HSA comprised 60% of the total protein mass of the plasma sample. PSV values of 0.733 mL/g were taken from the literature (Peters Jr, T., All about albumin: biochemistry, genetics, and medical applications, Academic press 1995).

A complex mixture, E. coli lysate (Bio-Rad) contained lyophilized E. coli cellular material of known mass. The lysate solution was meant to represent conditions comparable to unpurified lysates of expression systems. As Table 2 shows (row 5), despite the complex milieu that is the lysate, the peak height on the broad thermogram of the lysate alone, yielded a predicted mass (within 10%) of the actual mass of the lysate solution.

Purified and unpurified ACE2 were purchased from RayBiotech. The mass of purified ACE2 showed the largest difference between the measured and independently determined masses, but the absolute difference of (0.08 mg/mL) is really quite small. For the unpurified ACE2 in lysate, predicted mass concentrations of 0.04 mg/mL and 0.03 mg/mL were obtained for the C and N domains, respectively. When queried, the manufacturer did not know the concentration of ACE2 in the unpurified lysate. DSC analysis of unpurified ACE2 in the lysate background provided an estimate of that concentration (Table 2, row 6).

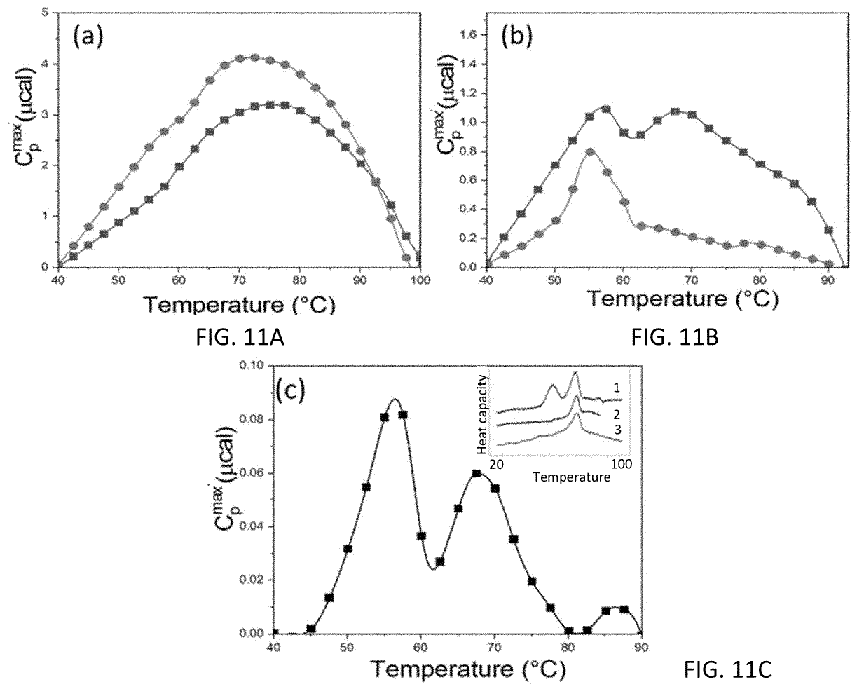

For proteins in complex media, the method was demonstrated to detect the proteins, isolate the thermogram for the protein of interest, and from that evaluate protein mass and effects of ligand binding. An example for human ACE2 is shown in FIGS. 11A-11C. This is a real world example of an expression system where ACE2 was overexpressed by HEK293 cells. For a comparative example, the extracted thermogram for ACE2 in complex media was compared with published thermograms for somatic bovine ACE2, FIG. 11C (inset). The nearly perfect agreement clearly demonstrates the feasibility of the method to detect both a target protein in complex media and also determine the mass concentration. Another successful example was demonstrated for streptavidin in E. coli lysate

Preliminary results demonstrated the ability to accurately evaluate mass concentrations for the proteins examined. However, the proteins examined were monomeric. ACE2 was the exception which is reportedly comprised of two monomeric subunits (Voronov et al., FEBS Letters 2002, 522:77-82). Using the disclosed method, a purification issue was identified. That is, while unpurified ACE2 displayed two peaks, purified ACE2 showed a single thermogram peak, in FIG. 9B, which provided an accurate mass for purified ACE2.

To demonstrate that the DNA standard can be universally applied to monomeric and multimeric proteins another known multimeric protein, streptavidin, was evaluated. Streptavidin is a tetrameric protein comprised of four identical subunits. Results for streptavidin were interesting and were compared to previous results in Table 2. Using the DNA standard, the protein mass was determined to be significantly greater than that evaluated by UV-absorbance. The ratio of the determined mass to the spectroscopic mass was 3.99. This result was extremely interesting since streptavidin with four domains has a determined mass exactly four times greater than the spectroscopic result. Provided further validation with other multimeric proteins can be obtained, application of the method can not only determine unknown mass concentrations of proteins in solution; it can also provide insights into quaternary structural composition of unknown proteins. It also can differentiate whether protein domains form from a contiguous primary structure i.e. monomeric (HSA, lysozyme); or are multimeric i.e. dimeric (unpurified ACE2) and tetrameric (streptavidin) which form via association of subunits.

Similar to streptavidin, the 16 base-pair double stranded DNA showed a discrepancy between the evaluated mass and the spectroscopic mass. In this case, the evaluated mass was nearly double (1.8) the spectrophotometrically determined mass.

### Example 4

### Relational Database Development and Use

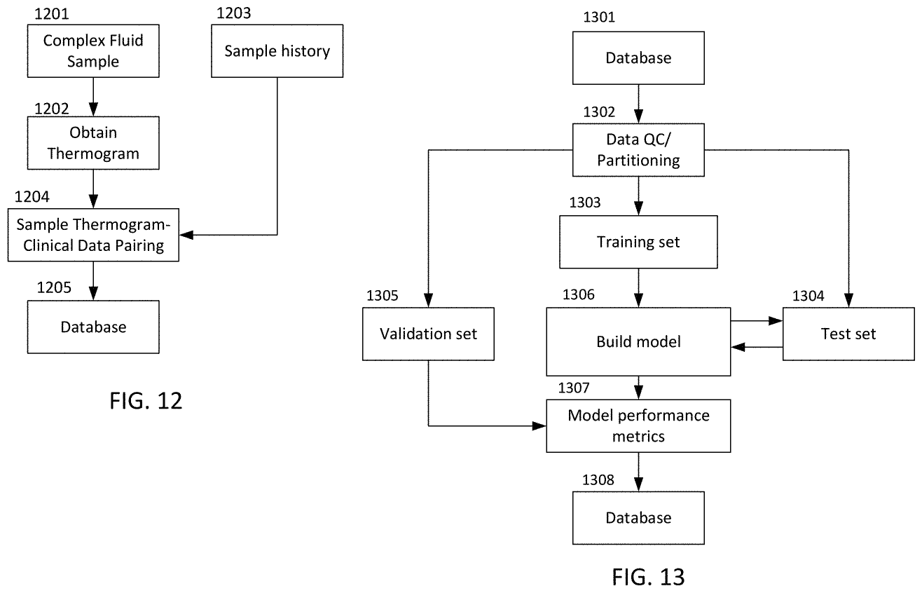

A generalized process for building a thermogram database is shown in FIG. 12. A complex fluid sample is obtained from a patient or prepared (1201). A thermogram of the sample is obtained (1202). Sample history (e.g., drug identification, patient status, etc.) is obtained (1203) and paired with the sample and the thermogram (1204). The paired data is transmitted to a computer database, such as a relational database (1205).

FIG. 13 illustrates an exemplary process for a machine-learning model. A database 1301 including thermograms and complex fluid sample data is provided. A data quality control and partitioning process 1302 is performed, and data is assigned to a training set of data 1303, a test set 1304 and/or a validation set 1305. The training set 1303 may be used to build a model 1306 including both complex fluid and thermogram data. Interactions between the test set 1304 and model 1306 are used to further develop the model. The validation set 1305 is utilized to determine model performance metrics 1307. The model performance metrics 1307 are included in the database 1308.

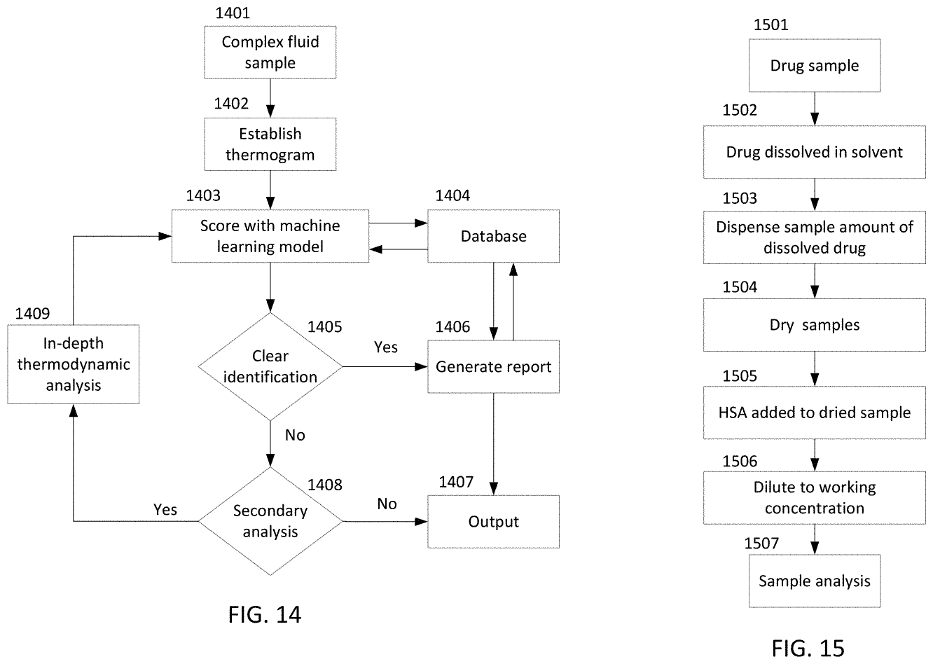

An exemplary process for evaluating, or scoring, complex fluid samples is illustrated in the flowchart of FIG. 14. A complex sample is obtained (1401), and a thermogram is established (1402). The thermogram is scored by the machine learning model (1403) and compared with data stored in the database (1404). An assessment of whether there is a clear identification of the target molecule(s) is made (1405). If the target molecule identification is clear, a report is generated (1406) and the report may be stored in the database (1404) and/or an output is generated (1407). If the target molecule identification is not clear, a decision is made whether to perform secondary analysis (1408). If no analysis is performed, an output is generated (1407). In some cases, an in-depth thermodynamic analysis is performed (1409) and the results are scored with the machine learning model (1403). The process then continues as described.

### Example 5

### Sample Preparation and Screening

An exemplary process for solubilizing drug samples is illustrated in the flowchart of FIG. 15. A drug sample (1501) is dissolved in a solvent (1502), such as an organic solvent. Sample amounts, or aliquots, of the dissolved drug are dispensed into vessels (1503), such as microcentrifuge tubes. The samples are then dried (1504), e.g., by vacuum concentration, to provide solid drug samples. The drug is combined with ligand, such as HSA (1505), and diluted to a final working concentration (1506) with a suitable solvent or buffer. Thermal analysis is then performed (1507).

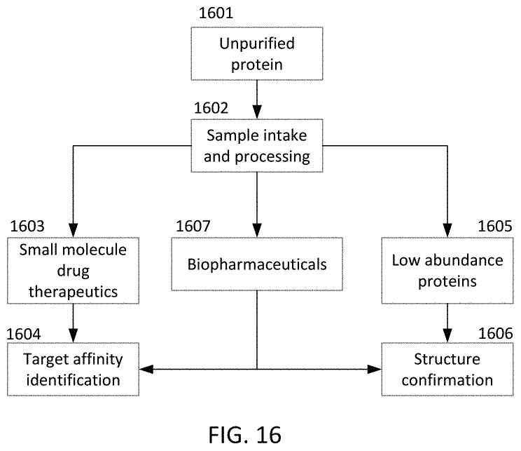

FIG. 16 illustrates an exemplary process for screening unpurified proteins (e.g., from a crude lysate). An unpurified protein sample is obtained (1601). A sample intake and processing is performed (1602). In some examples, the sample may be combined with small molecule drug therapeutics (1603), followed by thermal analysis to identify whether the unpurified protein has an affinity for the small molecule drug target and evaluation of PK/PD information (1604). In other examples, thermal analysis is performed to determine whether a low abundance protein is present in the unpurified protein sample (1605). Comparison of the thermogram to a reference library can provide rapid structure confirmation (1606) and, optionally, target affinity identification for the protein (1604). In still other examples, thermal analysis may be used to determine whether the protein is a biopharmaceutical candidate (1607). Comparison of the thermogram to a reference library can provide rapid structure confirmation (1606) and, optionally, target affinity identification with evaluation of PK/PD information for the biopharmaceutical candidate (1604).

### Example 6

### Infectious Disease Analysis

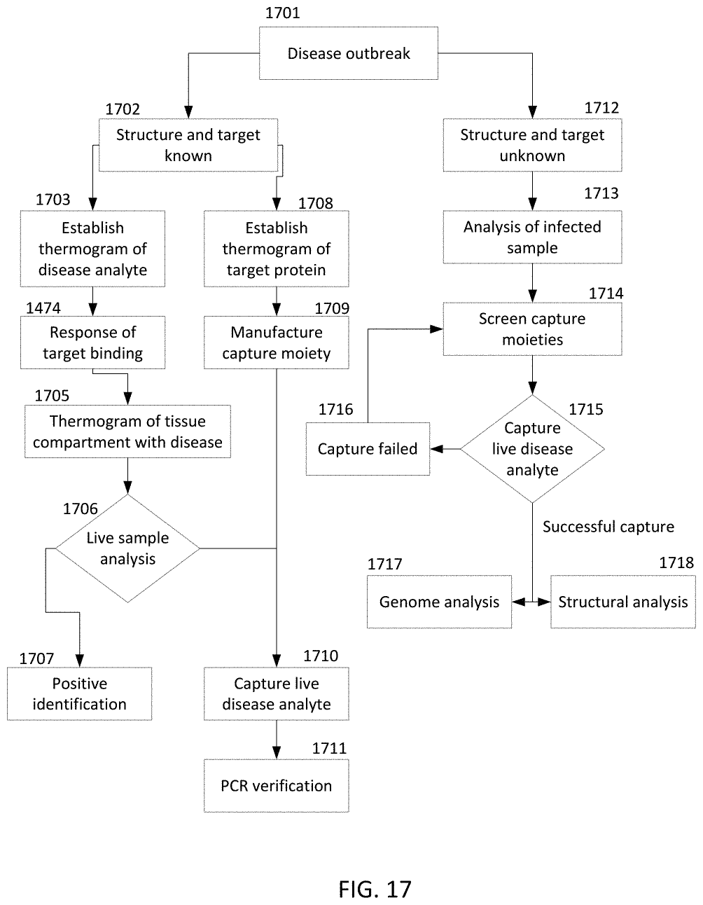

FIG. 17 illustrates one exemplary process for infectious disease analysis. A disease outbreak is detected (1701). In some instances, the disease analyte (e.g., a virus) structure and its target are known (1702). A thermogram of the disease analyte is established (1703) using a known sample including the disease analyte. An additional thermogram showing the effect produced by the disease analyte binding to its target is obtained (1704) using a known sample including the disease analyte and its target. Next a thermogram is established for a “live” sample obtained from a diseased tissue compartment (1705) and analysis of the live sample is performed (1706). The live sample analysis allows positive identification of the disease analyte (1707). In addition, or alternatively, a thermogram of the target protein (e.g., a target protein that binds with the disease analyte) is established (1708). Based at least in part on the thermogram results, a capture moiety is manufactured (1709). The capture moiety is combined with a live sample to capture the disease analyte (1710). If capture is successful, PCR verification may be used to confirm the disease analyte identity (1711).

In some instances, the disease analyte structure and target are unknown (1712). An infected sample is analyzed, e.g., by obtaining a thermogram (1713). One or more capture moieties is screened (1714) to determine whether it can capture the disease analyte (1715). If capture fails (1716), additional capture moieties are screened (1714, 1715). If capture is successful, genomic and/or structural analysis (1717, 1718) of the disease analyte is performed.

In view of the many possible embodiments to which the principles of the disclosed invention may be applied, it should be recognized that the illustrated embodiments are only preferred examples of the invention and should not be taken as limiting the scope of the invention. Rather, the scope of the invention is defined by the following claims. We therefore claim as our invention all that comes within the scope and spirit of these claims.

We claim:

1. A method, comprising:
(a) obtaining an analysis sample comprising a quantity of a complex fluid, the complex fluid comprising one or more of proteins, peptides, lipids, and carbohydrates, the complex fluid further comprising or suspected of comprising a target molecule;
(b) obtaining an analysis sample thermogram by differential scanning calorimetry;
(c) inputting the analysis sample thermogram into a computer system;
(d) comparing, using the computer system, the analysis sample thermogram to a (i) control sample thermogram, the control sample comprising the complex fluid, the control sample being devoid of the target molecule, (ii) one or more reference library thermograms of samples comprising known target molecules in the complex fluid, or both (i) and (ii) to provide a comparison;
(e) determining, using the computer system and based at least in part on the comparison, whether the analysis sample thermogram exhibits a perturbation; and
(f) if a perturbation is present, identifying the target molecule as present in the complex fluid.

2. The method of claim 1, wherein the target molecule comprises a protein, peptide, nucleic acid, lipid, carbohydrate, virus, or any combination thereof.

3. The method of claim 1, wherein the complex fluid is a biofluid, an environmental fluid, or any combination thereof.

4. The method of claim 1, wherein a perturbation is present, the method further comprising determining a mass of the target molecule in the complex fluid by:
adding a known amount of the target molecule or a standard molecule to the control sample to provide a known sample;
obtaining a known sample thermogram;
inputting the known sample thermogram into the computer system;
comparing, using the computer system, the known sample thermogram to the control sample thermogram;
determining, based at least in part on the comparison, a known perturbation measurement corresponding to the known amount of the target molecule or the standard molecule;
comparing the known perturbation measurement to a perturbation measurement corresponding to the target molecule in the analysis sample to provide a measurement comparison; and
determining, based at least in part on the measurement comparison, an amount of the target molecule in the analysis sample.

5. The method of claim 4, wherein:
(i) the standard molecule has a different composition than the target molecule; or
(ii) the standard molecule has a transition melting temperature of from 25° C. to 95° C.; or
(iii) the standard molecule comprises a DNA or RNA oligomer; or
(iv) the target molecule comprises a protein or a peptide; or
(v) any combination of (i), (ii), (iii), and (iv).

6. The method of claim 1 wherein the complex fluid comprises the target molecule, the method further comprising:
adding a quantity of a ligand to the analysis sample;
obtaining a subsequent analysis sample thermogram by differential scanning calorimetry;
inputting the subsequent analysis sample thermogram into the computer system;
comparing, using the computer system, the subsequent analysis sample thermogram to the analysis sample thermogram to provide a subsequent comparison; and
determining, using the computer system and based at least in part on the subsequent comparison, whether the subsequent analysis sample thermogram exhibits a subsequent perturbation, wherein a subsequent perturbation indicates binding of the ligand to the target molecule.

7. The method of 6, wherein a perturbation is present, the method further comprising:
determining, based at least in part on the perturbation, a characteristic of an interaction of the ligand with the target molecule, wherein the characteristic is a binding constant, reaction enthalpy, binding stoichiometry, binding free energy, binding entropy, or any combination thereof.

8. The method of claim 6, wherein the ligand comprises a protein, a peptide, a nucleic acid, a lipid, a carbohydrate, an organic small molecule having a molecular weight less than 1,000 daltons, a salt, an anion, a cation, a chelate, or any combination thereof.

9. The method of claim 8, wherein the ligand comprises an organic small molecule therapeutic agent, an antibody, a CAR (chimeric antigen receptor) T cell, a nucleic acid probe, a CRISPR (clustered regularly interspaced short palindromic repeats) product, or any combination thereof.

10. The method of claim 1, further comprising:
combining a quantity of a ligand with the analysis sample prior to obtaining the analysis sample thermogram, the ligand capable of binding to the target molecule; and
comparing, using the computer system, the analysis sample thermogram to (i) a control sample thermogram, the control sample comprising the complex fluid and the ligand, the control sample being devoid of the target molecule, (ii) a reference library of thermograms of samples comprising the ligand, or both (i) and (ii) to provide the comparison.

11. The method of claim 10, wherein a perturbation is present, the method further comprising determining an amount of the target molecule in the complex fluid by:
adding a known amount of a standard molecule to the control sample to provide a known sample;
obtaining a known sample thermogram;
inputting the known sample thermogram into the computer system;
comparing, using the computer system, the known sample thermogram to the control sample thermogram;
determining, based at least in part on the comparison, a known perturbation measurement corresponding to the known amount of the target molecule or the standard molecule;
comparing the known perturbation measurement to a perturbation measurement corresponding to the target molecule in the analysis sample to provide a measurement comparison; and
determining, based at least in part on the measurement comparison, an amount of the target molecule in the analysis sample.

12. The method of claim 1, wherein a perturbation is present, the method further comprising determining an amount of the target molecule in the complex fluid by:
determining, using the computer system and based at least in part on the perturbation, a perturbation measurement of the target molecule;
comparing, using the computer system, the thermodynamic melting parameter of the target molecule to a calibration curve comprising perturbation measurements of calibration solutions comprising varying amounts of a standard molecule, to provide a measurement comparison; and
determining, using the computer system and based at least in part on the measurement comparison, an amount of the target molecule in the analysis sample.

13. The method of claim 1, further comprising:
combining an additive with the analysis sample to provide a modified analysis sample, the additive comprising an inorganic salt, a protein, a carbohydrate, an amino acid, a vitamin, a peptide, a fatty acid, a lipid, a therapeutic agent, a solvent, or any combination thereof;
obtaining a modified analysis sample thermogram;
inputting the modified analysis sample thermogram into the computer system;
comparing, using the computer system, the modified analysis sample thermogram to the analysis sample thermogram; and
determining, using the computer system and based at least in part on the comparison, whether the modified analysis sample thermogram exhibits a perturbation relative to the analysis sample thermogram, wherein a perturbation indicates that the additive (i) altered a structure of the target molecule, (ii) altered an interaction of the ligand, if present, with the target molecule, or (iii) both (i) and (ii).

14. The method of claim 1, wherein obtaining the analysis sample further comprises:
combining, in a vessel, a capture moiety and a complex fluid comprising a target molecule or suspected of comprising a target molecule, the capture moiety comprising biotin covalently attached to a ligand capable of binding to the target molecule;
incubating the complex fluid and capture moiety whereby the target molecule, if present, binds to the capture moiety to form a conjugate;
removing the conjugate, if present, from the complex fluid with a device comprising (i) a body comprising a substrate material, a poly(methyl methacrylate) (PMMA) coating on at least a portion of a surface of the body, and a plurality of retrieval moiety molecules covalently bound to the PMMA coating, the retrieval moiety molecules comprising streptavidin;
removing the target molecule from the device; and
combining the removed target molecule with a quantity of a control sample to provide the analysis sample, wherein the control sample comprises the complex fluid, the control sample being devoid of the target molecule.

15. The method of claim 1, wherein:
the complex fluid comprises the target molecule in an unpurified state;
the control sample comprises the complex fluid and the target molecule in a purified state; and
a perturbation indicates that the target molecule in the purified state has an altered characteristic compared to the target molecule in the unpurified state.

16. The method of claim 15, further comprising:
combining a quantity of a ligand with the analysis sample prior to obtaining the analysis sample thermogram, the ligand capable of binding to the target molecule in the unpurified state;
comparing, using the computer system to a control sample thermogram, the control sample comprising the complex fluid, the target molecule added in the purified state, and the ligand to provide a subsequent comparison; and
determining, using the computer system and based at least in part on the subsequent comparison, whether the analysis sample exhibits a perturbation, wherein a perturbation indicates that the target molecule in the unpurified state exhibits different binding characteristics to the ligand compared to the target molecule in the purified state.

17. The method of claim 1, wherein the perturbation comprises:
(i) a change in height and/or width of a peak on the analysis sample thermogram relative to a corresponding peak on the control sample thermogram or reference library thermogram; or
(ii) a shift in position of a peak on the analysis sample thermogram relative to a corresponding peak on the control sample thermogram or reference library thermogram; or
(iii) presence of a peak on the analysis sample thermogram that is not present on the control sample thermogram or reference library thermogram; or
(iv) absence of a peak on the analysis sample thermogram compared to a peak that is present on the control sample thermogram or reference library thermogram; or
(v) any combination of (i), (ii), (iii), and (iv).

18. The method of claim 1, wherein:
the analysis sample is obtained from a subject;
the analysis sample comprises a quantity of a complex fluid comprising one or more of proteins, peptides, lipids, and carbohydrates, the complex fluid further comprising or suspected of comprising a virus;
the analysis sample thermogram is compared to a (i) control sample thermogram, the control sample comprising the complex fluid, the control sample being devoid of the virus, (ii) one or more reference library thermograms of samples comprising known viruses in the complex fluid, or both (i) and (ii) to provide a comparison; and
a perturbation indicates the virus is present in the complex fluid.

19. The method of claim 18, wherein a perturbation is present, the method further comprising:
combining a capture moiety with the analysis sample, the capture moiety comprising biotin covalently attached to a ligand capable of binding to the virus,
incubating the complex fluid and capture moiety whereby the virus binds to the capture moiety to form a conjugate,
removing the conjugate, if present, from the complex fluid with a device comprising (i) a body comprising a substrate material, a poly(methyl methacrylate) (PMMA) coating on at least a portion of a surface of the body, and a plurality of retrieval moiety molecules covalently bound to the PMMA coating, the retrieval moiety molecules comprising streptavidin, and
removing the virus from the device.

20. The method of claim 19, further comprising screening a ligand or a capture moiety to determine whether the ligand or capture moiety is capable of binding to the virus, wherein screening comprises:
combining the virus with a complex fluid devoid of the virus to provide a subsequent analysis sample;
adding a quantity of the ligand or capture moiety to the subsequent analysis sample;
obtaining a subsequent analysis sample thermogram by differential scanning calorimetry;
inputting the subsequent analysis sample thermogram into the computer system;
comparing, using the computer system, the subsequent analysis sample thermogram to a control sample thermogram, the control sample comprising the complex fluid and the virus, the control sample being devoid of the ligand or capture moiety; and
determining, using the computer system and based at least in part on the comparison, whether the subsequent analysis sample thermogram exhibits a subsequent perturbation, wherein a subsequent perturbation indicates binding of the ligand or capture moiety to the virus.

21. The method of claim 18, further comprising identifying the virus based at least in part on the perturbation, the subsequent perturbation, or the one or more genomic or structural analyses.

22. A method, comprising:
combining, in a vessel, (i) a complex fluid, comprising one or more of proteins, peptides, lipids, and carbohydrates, the complex fluid further comprising or suspected of comprising a target molecule, and (ii) a capture moiety comprising biotin covalently attached to a ligand capable of binding to the target molecule;
incubating the complex fluid and capture moiety whereby the target molecule, if present, binds to the capture moiety to form a conjugate;
removing the conjugate, if present, from the complex fluid with a device comprising (i) a body comprising a substrate material, a poly(methyl methacrylate) (PMMA) coating on at least a portion of a surface of the body, and a plurality of retrieval moiety molecules covalently bound to the PMMA coating, the retrieval moiety molecules comprising streptavidin;
removing the target molecule from the device;
combining the removed target molecule with a quantity of a control sample to provide an analysis sample, wherein the control sample comprises the complex fluid, the control sample being devoid of the target molecule;
obtaining an analysis sample thermogram by differential scanning calorimetry;
inputting the analysis sample thermogram into a computer system;
comparing, using the computer system, the analysis sample thermogram to (i) a control sample thermogram, the control sample comprising the complex fluid and the ligand, the control sample being devoid of the target molecule, (ii) a reference library of thermograms of samples comprising the ligand, or both (i) and (ii) to provide a comparison;
determining, using the computer system and based at least in part on the comparison, whether the analysis sample thermogram exhibits a perturbation; and
if a perturbation is present, identifying the target molecule as present in the complex fluid.


SEQUENCE LISTING (6 sequences)

SEQ ID NO: 1 - DNA, 20 bp, synthetic construct
  source 1..20: mol_type = other DNA
cgggcgcgttttcgcgcccg

SEQ ID NO: 2 - DNA, 12 bp, synthetic construct
  source 1..12: mol_type = other DNA
cggcttttgccg

SEQ ID NO: 3 - DNA, 20 bp, synthetic construct
  source 1..20: mol_type = other DNA
  misc_feature 5: note = n is any nucleotide except C
cgggngcgttttcgcgcccg

SEQ ID NO: 4 - DNA, 20 bp, synthetic construct
  source 1..20: mol_type = other DNA
cgggcgcgttttcgcgcccg

SEQ ID NO: 5 - DNA, 22 bp, synthetic construct
  source 1..22: mol_type = other DNA
cgggcgcgttttttcgcgcccg

SEQ ID NO: 6 - DNA, 20 bp, synthetic construct
  source 1..20: mol_type = other DNA
cgggcgcgttttcgcgcccg